SEQUENCE LISTING (70 sequences)

SEQ ID NO: 1 - protein, 133 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHP

SEQ ID NO: 2 - protein, 116 aa, synthetic construct
YIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLS

SEQ ID NO: 3 - protein, 19 aa, synthetic construct
ATNFSLLKQAGDVEENPGP

SEQ ID NO: 4 - protein, 133 aa, synthetic construct
RMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVL

SEQ ID NO: 5 - protein, 150 aa, synthetic construct
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYE

SEQ ID NO: 6 - protein, 94 aa, synthetic construct
YIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 7 - protein, 27 aa, synthetic construct
FWVLVVVGGVLACYSLLVTVAFIIFWV

SEQ ID NO: 8 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 130 aa, synthetic construct
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 10 - protein, 41 aa, synthetic construct
RSKRSRLLHSDYMNMTPRRPGPTRKHYQPYAPPRDFAAYRS

SEQ ID NO: 11 - protein, 55 aa, synthetic construct
KNRKAKAKPVTRGAGAGGRQRGQNKERPPPVPNPDYEPIRKGQRDLYSGLNQRRI

SEQ ID NO: 12 - protein, 45 aa, synthetic construct
GHETGRLSGAADTQALLRNDQVYQPLRDRDDAQYSHLGGNWARNK

SEQ ID NO: 13 - protein, 45 aa, synthetic construct
GQDGVRQSRASDKQTLLPNDQLYQPLKDREDDQYSHLQGNQLRRN

SEQ ID NO: 14 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLFQGLSTATKDTFDALHMQALPPR

SEQ ID NO: 15 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 16 - protein, 112 aa, synthetic construct
RVKFSRSADAPAYQQGQNQLFNELNLGRREEFDVLDKRRGRDPEMGGKPRRKNPQEGLFNELQKDKMAEAFSEIGMKGERRRGKGHDGLFQGLSTATKDTFDALHMQALPPR

SEQ ID NO: 17 - protein, 132 aa, synthetic construct
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVTTDSWGKLQFGAGTQVVVTP

SEQ ID NO: 18 - protein, 135 aa, synthetic construct
RMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRPGLAGGRPEQYFGPGTRLTVT

SEQ ID NO: 19 - protein, 134 aa, synthetic construct
METLLGLLILWLQLQWVSSKQEVTQIPAALSVPEGENLVLNCSFTDSAIYNLQWFRQDPGKGLTSLLLVRPYQREQTSGRLNASLDKSSGRSTLYIAASQPGDSATYLCAVRPGGAGSYQLTFGKGTKLSVIPN

SEQ ID NO: 20 - protein, 132 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPNMADEQYFGPGTRLTVTE

SEQ ID NO: 21 - protein, 134 aa, synthetic construct
RMSIGLLCCAALSLLWAGPVNAGVTQTPKFQVLKTGQSMTLQCAQDMNHEYMSWYRQDPGMGLRLIHYSVAIQTITDQGEVPNGYNVSRSTTEDFPLRLLSAAPSQTSVYFCASSYVGNTGELFFGEGSRLTVL

SEQ ID NO: 22 - protein, 133 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHP

SEQ ID NO: 23 - protein, 24 aa, Homo sapiens
IYIWAPLAGTCGVLLLSLVITLYC

SEQ ID NO: 24 - protein, 22 aa, Homo sapiens
MALIVLGGVAGLLLFIGLGIFF

SEQ ID NO: 25 - protein, 21 aa, Homo sapiens
LCYLLDGILFIYGVILTALFL

SEQ ID NO: 26 - protein, 116 aa, Homo sapiens
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 27 - protein, 52 aa, Homo sapiens
YFLGRLVPRGRGAAEAATRKQRITETESPYQELQGQRSDVYSDLNTQRPYYK

SEQ ID NO: 28 - protein, 61 aa, Homo sapiens
NRRRRRERRDLFTESWDTQKAPNNYRSPISTSQPTNQSMDDTREDIYVNYPTFSRRPKTRV

SEQ ID NO: 29 - protein, 111 aa, synthetic construct
FWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 30 - protein, 42 aa, synthetic construct
KRGRKKLLYIFKQPFMRPVQTTQEEDGCSCRFPEEEEGGCEL

SEQ ID NO: 31 - protein, 38 aa, synthetic construct
CWLTKKKYSSSVHDPNGEYMFMRAVNTAKKSRLTDVTL

SEQ ID NO: 32 - protein, 42 aa, Homo sapiens
RLKIQVRKAAITSYEKSDGVYTGLSTRNQETYETLKHEKPPQ

SEQ ID NO: 33 - protein, 77 aa, Homo sapiens
CRKKRISANSTDPVKAAQFEPPGRQMIAIRKRQLEETNNDYETADGGYMTLNPRAPTDDDKNIYLTLPPNDHVNSNN

SEQ ID NO: 34 - protein, 61 aa, Homo sapiens
RKRWQNEKLGLDAGDEYEDENLYEGLNLDDCSMYEDISRGLQGTYQDVGSLNIGDVQLEKP

SEQ ID NO: 35 - protein, 49 aa, Homo sapiens
LDKDDSKAGMEEDHTYEGLDIDQTATYEDIVTLRTGEVKWSVGEHPGQE

SEQ ID NO: 36 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 37 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 38 - protein, 45 aa, synthetic construct
CDVKLVEKSFETDTNLNFQNLSVIGFRILLLKVAGFNLLMTLRLW

SEQ ID NO: 39 - protein, 41 aa, synthetic construct
CGFTSESYQQGVLSATILYEILLGKATLYAVLVSALVLMAM

SEQ ID NO: 40 - protein, 16 aa, synthetic construct
  VARIANT 11..12: note = 1 or 2 amino acids may be absent
  VARIANT 2..15: note = X can be any amino acid
YXXJXXXXXXXXYXXJ

SEQ ID NO: 41 - skipped

SEQ ID NO: 42 - protein, 16 aa, synthetic construct
  VARIANT 11..12: note = 1 or 2 amino acids may be absent
  VARIANT 2..15: note = X can be any amino acid
FXXJXXXXXXXXFXXJ

SEQ ID NO: 43 - protein, 15 aa, Homo sapiens
CGFTSVSYQQGVLSA

SEQ ID NO: 44 - protein, 15 aa, Homo sapiens
CGFTSESYQQGVLSA

SEQ ID NO: 45 - protein, 22 aa, synthetic construct
CDVKLVEKSFETDTNLNFQNLS

SEQ ID NO: 46 - protein, 15 aa, synthetic construct
  VARIANT 6: note = X is valine or glutamic acid
CGFTSXSYQQGVLSA

SEQ ID NO: 47 - protein, 23 aa, synthetic construct
VIGFRILLLKVAGFNLLMTLRLW

SEQ ID NO: 48 - protein, 18 aa, synthetic construct
ILLGKATLYAVLVSALVL

SEQ ID NO: 49 - protein, 23 aa, synthetic construct
VIGFRILLLKVAGFNLLMTLRLW

SEQ ID NO: 50 - protein, 12 aa, synthetic construct
SSGSRAKRSGSG

SEQ ID NO: 51 - protein, 21 aa, synthetic construct
ILLGKATLYAVLVSALVLMAM

SEQ ID NO: 52 - protein, 8 aa, synthetic construct
VKRKDSRG

SEQ ID NO: 53 - protein, 615 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKRSGSGATNFSLLKQAGDVEENPGPRMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 54 - protein, 790 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPRMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 872 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPRMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 366 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 401 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 407 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 442 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 366 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 407 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 367 aa, synthetic construct
METLLGLLILWLQLQWVSSKQEVTQIPAALSVPEGENLVLNCSFTDSAIYNLQWFRQDPGKGLTSLLLVRPYQREQTSGRLNASLDKSSGRSTLYIAASQPGDSATYLCAVRPGGAGSYQLTFGKGTKLSVIPNYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 401 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPNMADEQYFGPGTRLTVTEEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 408 aa, synthetic construct
METLLGLLILWLQLQWVSSKQEVTQIPAALSVPEGENLVLNCSFTDSAIYNLQWFRQDPGKGLTSLLLVRPYQREQTSGRLNASLDKSSGRSTLYIAASQPGDSATYLCAVRPGGAGSYQLTFGKGTKLSVIPNYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 442 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPNMADEQYFGPGTRLTVTEEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - skipped

SEQ ID NO: 67 - protein, 151 aa, synthetic construct
YIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKRSGSG

SEQ ID NO: 68 - protein, 179 aa, synthetic construct
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 69 - protein, 21 aa, Homo sapiens
ILLGKATLYAVLVSALVLMAM

SEQ ID NO: 70 - protein, 23 aa, Homo sapiens
TILYEILLGKATLYAVLVSALVL

(19) United States
(12) Patent Application Publication
(10) Pub. No.: US 2026/0315530 A1
(43) Pub. Date: Oct. 15, 2026

(54) PMHC-BINDING HETERODIMERIC RECEPTORS WITH AN IMPROVED DISCRIMINATION BETWEEN A LOW AFFINITY AND A HIGH AFFINITY PMHC AND THAT DO NOT ASSOCIATED WITH CD3

(71) Applicant: Oxford University Innovation Limited, Oxford (GB)

(72) Inventor: Omer DUSHEK, Oxford (GB)

(21) Appl. No.: 19/117,235

(22) PCT Filed: Aug. 9, 2023
(86) PCT No.: PCT/GB2023/052101

§ 371 Date: Mar. 31, 2025

(30) Foreign Application Priority Data
Oct. 14, 2022 (GB) 2215233.4

(51) Int. Cl. A61K 35/17; A61K 31/7088; A61K 38/00; A61K 40/11; A61K 40/32; A61K 40/42; A61P 31/00; C07K 14/705; C07K 16/00; C07K 19/00; C12N 5/0783; C12N 15/63; G01N 33/68

(52) U.S. Cl. CPC A61K 35/17; A61K 31/7088; A61K 38/00; A61K 40/11; A61K 40/32; A61K 40/42; A61P 31/00; C07K 14/7051; C07K 14/70517; C07K 14/70521; C07K 14/70539; C07K 16/00; C07K 19/00; C12N 5/0638; C12N 15/63; G01N 33/68; C07K 2317/52; C07K 2317/565; C07K 2319/02; C07K 2319/03; G01N 2333/705

(57) ABSTRACT

The invention relates to heterodimeric pMHC-binding receptors with enhanced discrimination for their target pMHC.

### FIELD OF INVENTION

The invention relates to receptors specific for major histocompatibility complex (MHC)-restricted peptide antigens, and methods and uses thereof.

### BACKGROUND OF THE INVENTION

Many T cell therapies rely on introduction of heterologous T cell receptors (TCRs) or modified autologous TCRs into a patient to exploit novel, high affinity binding interactions between the TCR and a pathological peptide presented via the major histocompatibility complex (pMHC). These TCRs are optimised, validated and screened for positive target and negative off-target binding in vitro. Testing in animal models is rare, since TCRs are designed with human immunological compatibility in mind, so the first in vivo administration of therapeutic TCRs or therapeutic T cells bearing said TCRs is usually in human clinical trials.

Exogenously introduced therapeutic TCRs bypass thymic selection and functional central tolerance processes, increasing the risk of off-target interactions with self antigens. Although these off-target interactions with self antigens are expected to be of lower affinity, it has recently been shown that TCR antigen discrimination based on affinity is imperfect: primary human T cells can respond to pMHC with affinities as low as KD~1 mM (1). Therefore, although the affinity between introduced TCRs and off-target self antigens is expected to be low, it may be functionally significant in vivo and can result in serious, and even fatal, side effects. For example, fatalities in the introduced A3A TCR clinical trial, whose on-target antigen was MAGE-A3, have been attributed to off-target binding of the A3A TCR to the self-antigen Titin (2,3), which were not identified in extensive in vitro safety screens due to a combination of low affinity binding and lack of surface expression on in vitro cultured cells.

Strategies exist to minimise the risk of self antigen interaction, but these have limitations. In vitro screens for low affinity off-target binding are labour-intensive and expensive, and do not achieve full coverage of tissue-, differentiation- and patient-specific self-peptide repertoires. Computational prediction and artificial intelligence (AI)-based strategies can be employed, but the small and variable number of residue contact points between the TCR (at the CDR3α and CDR3β loops) and antigen render this highly complex.

It is an object of the invention to provide further and improved pMHC-binding receptors that when expressed in T cells allow responses to higher-affinity on-target antigens but not to lower-affinity off-target antigens.

### SUMMARY OF THE INVENTION

The inventors found that discrimination for target MHC-restricted peptide antigens (‘pMHCs’) can be enhanced using a novel TCR-like heterodimeric architecture as described herein.

Surprisingly, the inventors found that modifying TCRs to abrogate interaction with the CD3 complex enhances the capacity of the pMHC-binding receptor to discriminate between low and high affinity pMHCs. Additional benefits of a lack of association with the CD3 complex include: (i) increased expression levels of the pMHC-binding receptor in T cells, since the pMHC-binding receptor does not compete with endogenous TCRs for limited CD3; (ii) directed heterodimerisation, since the α and β chains of pMHC-binding receptors are unable to dimerise with endogenous TCRα and TCRβ to form unwanted TCR combinations with potential novel cross-reactivities; (iii) the pMHC-binding receptors can be transduced into immune cells other than T cells, since they are not dependent on CD3 expression for function.

The modified TCRs, also referred herein as pMHC-binding receptor or enhanced discrimination receptors (‘EDRs’), are incapable of association with the CD3 complex. In a conventional TCR, αβTCR:CD3 complexation is mediated via highly conserved residues within the extracellular and transmembrane portions of the α and β chains (4). The EDRs of the invention delete or replace the relevant portions of the α and β chain sequences with non-TCR-derived transmembrane regions such that association with the CD3 complex is lost. Loss of CD3 complexation would typically prevent surface expression of the αβ chains due in part to exposure of the hydrophobic residues within connecting peptide motifs (CPMs) in the membrane-proximal portion of the extracellular region. Removing these CPMs enabled efficient surface expression of EDRs without the CD3 complex.

In particular, the TCR was modified such that the receptor is a heterodimer formed by two polypeptides, with each polypeptide comprising an extracellular portion, a transmembrane portion, and an intracellular portion, and the extracellular portions of the two polypeptides form a complex to create the pMHC-binding portion of the resulting receptor.

For example, the first polypeptide of the dimer may comprise, from N-terminus to C-terminus, a TCR variable alpha domain coupled to (or grafted on to) a truncated TCR constant alpha region that does not contain the CPM that associates with CD3, a transmembrane region (e.g. from CD2, CD8alpha, CD4 or CD247), and a signalling tail which usually contains an ITAM domain. The second polypeptide may be the same as the first polypeptide except the second polypeptide comprises a TCR beta variable domain coupled to a truncated constant region that does not contain the CPM that associates with CD3, instead of the TCR variable and constant alpha regions. Upon dimerisation of the first and second polypeptides, the TCR variable alpha domain from the first polypeptide and the TCR variable beta domain from the second polypeptide form an pMHC-binding portion corresponding to that of the parent TCR. The truncated TCR constant alpha region from the first polypeptide and the truncated TCR constant beta region from the second polypeptide form a constant region similar to that of the parent TCR but lacking the CPMs.

Accordingly, the invention provides MHC-presented peptide (pMHC)-binding receptor comprising two polypeptides forming a heterodimer which comprises: (a) an extracellular portion comprising a pMHC-binding portion; (b) a transmembrane portion; and (c) an intracellular portion comprising a signalling domain.

The invention also provides MHC-presented peptide (pMHC)-binding receptor comprising two polypeptides forming a heterodimer which comprises: (a) an extracellular portion comprising a pMHC-binding portion derived from a T cell receptor (TCR); (b) a transmembrane portion; and (c) an intracellular portion comprising a signalling domain.

The invention also provides one or more isolated polynucleotides encoding the first polypeptide and/or second polypeptide of the pMHC-binding receptor described herein.

The invention also provides one or more expression constructs comprising one or more polynucleotides described herein.

The invention also provides one or more vectors comprising the one or more polypeptides or the one or more expression constructs described herein.

The invention also provides a cell comprising one or more polynucleotides, one or more expression constructs, or one or more vectors described herein.

The invention also provides a cell comprising a pMHC-binding receptor described herein.

The invention also provides a pharmaceutical composition comprising: (i) one or more polynucleotides, expression constructs, or vectors described herein, and (ii) a pharmaceutically acceptable carrier.

The invention also provides a pharmaceutical composition comprising a cell described herein and a pharmaceutically acceptable carrier.

The invention also provides a pMHC-binding receptor, one or more polynucleotides, expression constructs, vectors, a cell or a pharmaceutical composition described herein for use as a medicament, such as for use in treating cancer or an infection (e.g. a chronic infection).

The invention also provides the use of a pMHC-binding receptor, one or more polynucleotides, expression constructs, vectors, a cell, or a pharmaceutical composition described herein in the manufacture of a medicament for the treatment of cancer or an infection (e.g. a chronic infection) or an inflammatory disease (e.g. autoimmunity).

The invention also provides a method of treating cancer or an infection (e.g. a chronic infection) or an inflammatory disease comprising administering a pMHC-binding receptor, one or more polynucleotides, expression constructs, vectors, a cell or a pharmaceutical composition described herein to a patient in need thereof.

The invention also provides the use of the pMHC-binding receptor, one or more polynucleotides, expression constructs, vectors, a cell, or a pharmaceutical composition described herein to treat cancer or an infection (e.g. a chronic infection) or an inflammatory disease.

The invention also provides a method of identifying a pMHC-binding receptor specific to a target pMHC of interest, wherein the method comprises screening a library of pMHC-binding receptors described herein for high affinity binding to said target pMHC.

The invention also provides a method of identifying low-affinity or ‘off-target’ binding interactions comprising screening a library of MHC-presented peptides (pMHCs) for binding to a pMHC-binding receptor described herein, optionally wherein said library does not comprise the pMHC-binding receptor's target pMHC.

The invention also provides a method of assessing discrimination capacity of a pMHC-binding receptor described herein, comprising exposing the pMHC-binding receptor to on-target higher affinity and one or more off-target low affinity pMHCs (i.e. pMHC for which it is not specific) and comparing the concentration of pMHC required to induce activation of cells expression the pMHC-binding receptor, wherein a large difference in concentration indicates good discrimination.

The invention also provides a molecule obtainable by a method described herein.

The invention also provides a method, such as an ex vivo method, of preparing a population of immune cells for adoptive cell therapy, the method comprises culturing an immune cell described herein to produce a population of immune cells.

The invention also provides a population of immune cells produced by a method described herein.

These and further aspects of the invention are described in more detail herein.

### BRIEF DESCRIPTION OF THE FIGURES

FIG. 1. Schematics of (A) the T cell receptor (TCR) and (B) an Enhanced Discriminatory Receptor (EDR). The EDR (i.e. a pMHC-binding receptor of the invention) as shown in the figure is a heterodimeric dimer comprising a TCR alpha-chain and a TCR beta-chain that each contains an extracellular variable and constant region, and each chain is coupled to a transmembrane domain, and intracellular signalling domains.

FIG. 2. Surface expression of the EDR does not require CD3 surface expression. E6.1 TCRα⁻β⁻ Jurkat cells were lentivirally transduced with either the 1G4 TCR or a 1G4 EDR. PE-Tetramers of the 9V pMHC antigen detects surface levels of each antigen receptor (A) and UCHT antibody detected surface CD3E levels (B). The 1G4 EDR was detected at the cell surface using pMHC tetramers without CD3E upregulation indicating these receptors are CD3-independent, which is not the case for the 1G4 TCR.

FIG. 3. Surface expression of the EDR and TCR on primary human T cells. PE-Tetramers of the 9V pMHC antigen detected surface levels of each antigen receptor on primary human CD8⁺ T cells lentivirally transduced with the 1G4 TCR or the 1G4 EDR.

FIG. 4. The EDR exhibits enhanced antigen discrimination compared to the TCR when measuring surface CD69. Primary human CD8+ T cells transduced with the 1G4 TCR (circles) or 1G4 EDR (squares) were stimulated by Nalm6 target cells loaded with the indicated concentration of peptide antigen (x-axis) and surface CD69 was measured using flow cytometry after 20 hours (y-axis). Data is shown for the indicated peptide antigens: (A) 9V, (B) 6V, (C) 3Y, (D) 6T, (E) 4D, (F) 4A, and (G) 5Y. Representative data is shown from 1 out of 3 independent experiments.

FIG. 5. The EDR requires a higher concentration of lower-affinity but not higher-affinity antigens to induce T cell activation compared to the TCR when measuring surface CD69. (A) The concentration of antigen required to activate 15% of T cells above background (P15) from the data in FIG. 4 is plotted over the TCR/pMHC affinity measured using SPR. The affinity measurements were previously reported (1). (B) The fold-change in P15 between the TCR and EDR from panel A is plotted over the TCR/pMHC affinity showing that a similar antigen concentration is required to activate T cells through the TCR and EDR for higher-affinity antigens (e.g. 9V) but a larger antigen concentration (>20-fold) is required to activate T cells through the EDR compared to the TCR for lower-affinity antigens (e.g. 4D).

FIG. 6. The EDR exhibits enhanced antigen discrimination compared to the TCR when measuring IL-2 production. Primary human CD8+ T cells transduced with the 1G4 TCR (circles) or 1G4 EDR (squares) were stimulated by Nalm6 target cells loaded with the indicated concentration of peptide antigen (x-axis) and supernatant TL-2 was measured using ELISA after 20 hours (y-axis). Data is shown for the indicated peptide antigens: (A) 9V, (B) 6V, (C) 3Y, (D) 6T, (E) 4D, (F) 4A, and (G) 5Y. Representative data is shown from 1 out of 3 independent experiments.

FIG. 7. The EDR requires a higher concentration of lower-affinity but not higher-affinity antigens to induce T cell activation compared to the TCR when measuring IL-2 production. (A) The concentration of antigen required to activate 15% of T cells above background (P15) from the data in FIG. 6 is plotted over the TCR/pMHC affinity measured using SPR. The affinity measurements were previously reported (1). (B) The fold-change in P15 between the TCR and EDR from panel A is plotted over the TCR/pMHC affinity.

FIG. 8. The EDR exhibits enhanced antigen discrimination compared to the TCR when measuring CD69 or IL-2 on the U87 glioblastoma cell line. Data generated as described in FIG. 4,5,6,7 except using the U87 glioblastoma target cell line. (A-D) Surface CD69 and (E-H) Supernatant IL-2 for the indicated peptide antigens. (I-J) The concentration of peptide antigen required to elicit 15% activation above baseline (P15) over the antigen affinity for (I) Surface CD69 or (J) supernatant IL-2. (A-H) Representative example (I-J) mean+/−SEM for N=3 independent repeats. The p-value for the null hypothesis that the P15 is the same between the TCR and EDR was carried out on log-transformed values using a paired t-test. Abbreviations: p<0.05 (*), p<0.01 (**), p<0.005 (***).

FIG. 9. An EDR with CD28 co-stimulation performs similarly to the TCR. Primary human CD8+ T cells transduced with the 1G4 TCR (circles) or 1G4 EDR (squares) were stimulated for 20 hours by T2 target cells loaded with the indicated concentration of 9V peptide antigen (x-axis) with flow cytometry used to measure (A) surface 4-1BB and (B) surface CD25 and ELISA used to measure (C) IL-2 and (D) MIP-1b in the supernatant.

FIG. 10. An EDR generated from the A6 TCR that recognises the Tax peptide from HTLV displays higher surface expression and antigen sensitivity. (A) Surface expression of the A6 TCR or the A6 EDR on primary human CD8+ T cells detected with PE-tetramers of the Tax pMHC. (B) Indicated cytokine production (IFNγ, IL-a and MIP-1β) by primary human CD8+ T cells transduced with the A6 TCR (circles) or A6 EDR (squares) stimulated by U87 glioblastoma target cells loaded with the indicated concentration of the wild-type Tax peptide antigen.

FIG. 11. Antigen discrimination by the A6 EDR performs similarly to the 1G4 EDR and displays higher discrimination compared to the TCR. (A) Production of the cytokine IL-2 by primary human CD8+ T cells transduced by the A6 EDR and stimulated with U87 glioblastoma target cells loaded with the indicated concentration of the indicated peptide antigens. (B) The concentration of antigen required to produce IL-2 at 15% above background (P15) from the data in FIG. 11A over the antigen affinity (K_{D}) for the A6 EDR (diamonds). For comparison, data is shown for the 1G4 EDR (squares) and 1G4 TCR (circles) from FIG. 7A. The values of K_{D} for the different antigens were measured previously (1). (C) The relationship between P15 and K_{D} in FIG. 11B can be fit by a power law, P15=constant (K_{D})^{α}, where α is defined as the discrimination power with larger values indicating improved discrimination. The discrimination power (α) is the slope of the line in panel B (i.e. when both axes are logarithmic).

FIG. 12. Schematic of the design of a pMHC-binding receptor of the invention. A pMHC-binding receptor of the invention (“TCR-based EDR”) may comprise an extracellular domain that is derived from a T cell receptor (TCR). Hence, the EDR may comprise the TCR variable α and β domains (Vα and Vβ), and truncated constant a and R regions (Cα and Cβ) as described herein. In another embodiment, a pMHC-binding receptor of the invention (“antibody-based EDR”) may comprise an extracellular portion that is derived from an antibody. Hence, the antibody-based EDR may comprise the heavy and light chain variable domains (V_{H} and V_{L}), a light chain constant domain (C_{L}) and one of the heavy chain constant domains, e.g. C_{H1}, of a pMHC-binding antibody. The pMHC-binding receptor of the invention has minimal interaction with the endogenous CD3 components.

FIG. 13. Raw cytokine secretion by T cells expressing the 1G4 TCR or 1G4 EDR in response to cells expressing random peptide mixtures of different lengths. TAP-deficient, HLA-A2:01+T2 cells were loaded with random peptide libraries of different lengths (X₁X₂ . . . X_{N}, where X is any amino acid except cysteine and N is the length). Primary human CD8+ T cells transduced with the 1G4 TCR, 1G4-z/z-EDR, or untransduced were co-cultured with the T2 cells for 24 h, and the supernatants assayed for the production of GM-CSF, MIP-1β, and TNF-α. Co-cultures were conducted using 100 μM concentration of the relevant peptide mixture, and at a gross E:T ratio of 1:1. Data is shown for 3 independent experiments (donors) each carried out with 7 technical replicates. Data for donor 1 are shown in panels A-C, donor 2 in panels D-F and donor 3 in panels G-I.

FIG. 14. Combined cytokine secretion by T cells expressing the 1G4 TCR or 1G4 EDR in response to cells expressing random peptide mixtures of different lengths. The total amount of secreted cytokine, calculated by adding the cytokine secreted from the technical replicates in FIG. 13, for the three independent experiments for the indicates library length for (A) GM-CSF, (B) MIP-1β, and (C) TNF-α. The addition was conducted after subtraction of the baseline DMSO response.

FIG. 15. A T cell cross-reactivity index shows that the 1G4 EDR has lower cross-reactivity compared to the 1G4 TCR. The combined cytokine secretion from FIG. 14 for each donor was normalised to the response of the N=9 random library and then the average normalised response across all library lengths was computed to produce the T-cell Cross-reactivity Index for (A) GM-CSF, (B) MIP-1β, and (C) TNF-α. Each data point corresponds to an individual donor. Statistical comparisons were conducted using an unpaired t test (ns=not significant, **=p<0.01).

### DETAILED DESCRIPTION OF THE INVENTION

The invention relates to a heterodimeric pMHC-binding receptor comprising: (a) an extracellular portion comprising a pMHC-binding portion; (b) a transmembrane portion; and (c) an intracellular portion. Said ‘extracellular’, ‘transmembrane’ and ‘intracellular’ portions are so-called due to their respective locations when the receptor is expressed and assembled within a cell or cell-like structure (for example a lipid nanoparticle or other membranous structure which resembles a phospholipid bilayer).

Receptors of the invention are heterodimeric in that they comprise a first and a second polypeptide (also referred to herein as a first and second ‘polypeptide chain’). The first polypeptide and the second polypeptide may each comprise (a) an extracellular portion; (b) a transmembrane portion; and/or (c) an intracellular portion. Typically, the first polypeptide and the second polypeptide each comprises (a) an extracellular portion; (b) a transmembrane portion; and (c) an intracellular portion.

Upon dimerisation of the first and second polypeptides, parts of the extracellular portions of the first and second polypeptides may form the pMHC-binding portion in the extracellular portion of the pMHC-binding receptor. Upon dimerisation of the first and second polypeptides, parts of the transmembrane portions of the first and second polypeptides may form the transmembrane portion of the pMHC-binding receptor. Upon dimerisation of the first and second polypeptides, parts of the intracellular portions of the first and second polypeptides may form the intracellular portion of the pMHC-binding receptor.

The amino acid sequences of the first and second polypeptides are different. The first and second polypeptides associate post-translationally. The association may be non-covalent binding. The association may be substantially non-covalent binding. The association may comprise an interchain covalent interaction, such as an interchain disulphide bond.

### Extracellular Portion

The extracellular portion of a receptor of the invention comprises a pMHC-binding portion which confers specificity for a peptide antigen presented on a MHC molecule. The pMHC-binding portion is typically at the membrane-distal portion of the extracellular portion of the receptor.

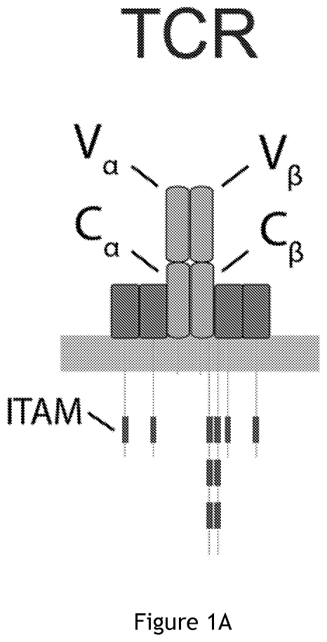

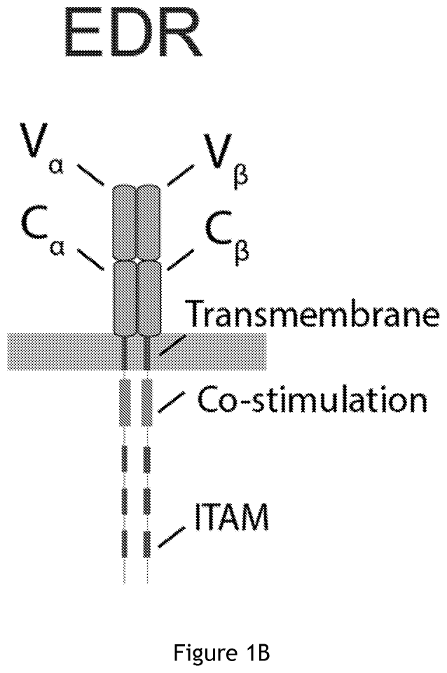

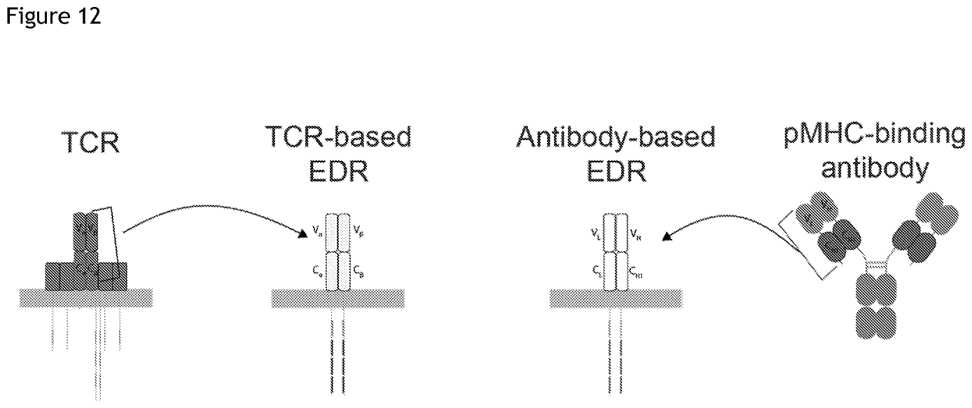

The pMHC-binding portion may be derived from a T cell receptor (TCR), i.e. the pMHC-binding portion of a receptor of the invention may comprise the pMHC-binding portion of a TCR (e.g. see FIGS. 1B and 12).

The pMHC-binding portion may comprise a CDR3α loop and a CDR3β loop and may comprise a CDR1α loop, a CDR1β loop, a CDR2α loop, a CDR2β loop, a CDR3α loop and a CDR3β loop. The pMHC-binding portion may comprise a TCR α variable domain and a TCR β variable domain.

For example, pMHC-binding portion may comprise the pMHC binding portion of a TCR specific for NY-ESO-1/MHC (e.g. HLA-A*02) complex. For example, the pMHC-binding portion may comprise a TCR α variable domain as set out in SEQ ID NO: 1 and TCR β variable domain as set out in SEQ ID NO: 4. The pMHC-binding portion may comprise a TCR α variable domain comprising an amino acid sequence having at least 70%, at least 80%, at least 90%, at least 95%, at least 98%, at least 99% sequence identity to SEQ ID NO: 1, wherein the pMHC-binding portion is capable of binding to NY-ESO-1/MHC; and a TCR β variable domain comprising an amino acid sequence having at least 70%, at least 80%, at least 90%, at least 95%, at least 98%, at least 99% or 100% sequence identity to SEQ ID NO: 4, wherein the pMHC-binding portion is capable of binding to NY-ESO-1/MHC.

The pMHC-binding portion may comprise the pMHC binding portion of a TCR specific for Tax/MHC (e.g. Tax/HLA-A*02) complex. For example, the pMHC-binding portion may comprise a TCR α variable domain comprising an amino acid sequence as set out in SEQ ID NO: 17 and a TCR β variable domain comprising an amino acid sequence as set out in SEQ ID NO: 18 The pMHC-binding portion may comprise a TCR α variable domain comprising an amino acid sequence having at least 70%, at least 80%, at least 90%, at least 95%, at least 98% or at least 99% sequence identity to SEQ ID NO: 17, wherein the pMHC-binding portion is capable of binding to Tax/MHC; and a TCR β variable domain comprising an amino acid sequence having at least 70%, at least 80%, at least 90%, at least 95%, at least 98% or at least 99% sequence identity to SEQ ID NO: 18, wherein the pMHC-binding portion is capable of binding to Tax/MHC.

The pMHC-binding portion may comprise the pMHC binding portion of a TCR specific for MAGE-A3/MHC (e.g. MAGE-A3/HLA-A*01) complex. For example, the pMHC-binding portion may comprise a TCR α variable domain comprising an amino acid sequence as set out in SEQ ID NO: 19 and a TCR β variable domain comprising an amino acid sequence as set out in SEQ ID NO: 20. The pMHC-binding portion may comprise a TCR α variable domain comprising an amino acid sequence having at least 70%, at least 80%, at least 90%, at least 95%, at least 98% or at least 99% sequence identity to SEQ ID NO: 19, wherein the pMHC-binding portion is capable of binding to MAGE-A3/MHC; and a TCR β variable domain comprising an amino acid sequence having at least 70%, at least 80%, at least 90%, at least 95%, at least 98% or at least 99% sequence identity to SEQ ID NO: 20, wherein the pMHC-binding portion is capable of binding to MAGE-A3/MHC). The pMHC-binding portion may comprise the pMHC binding portion of a TCR specific for NY-ESO-1/MHC (e.g. HLA-A*02) (wt/c51). For example, the pMHC-binding portion may comprise a TCR α variable domain comprising an amino acid sequence as set out in SEQ ID NO: 1 and a TCR β variable domain comprising an amino acid sequence as set out in SEQ ID NO: 21. The pMHC-binding portion may comprise a TCR α variable domain comprising and amino acid sequence having at least 70%, at least 80%, at least 90%, at least 95%, at least 98% or at least 99% sequence identity to SEQ ID NO: 1, wherein the pMHC-binding portion is capable of binding to NY-ESO-1/MHC; and a TCR β variable domain comprising an amino acid sequence having at least 70%, at least 8000, at least 90%, at least 95%, at least 98% or at least 99% sequence identity to SEQ ID NO: 21 wherein the pMHC-binding portion binds NY-ESO-1/MHC.

The pMHC-binding portion may comprise the pMHC binding portion of a TCR specific for NY-ESO-1/MHC (e.g. HLA-A*02) (C259/wt). For example, the pMHC-binding portion may comprise a TCR α variable domain comprising an amino acid sequence as set out in SEQ ID NO: 22 and a TCR β variable domain comprising an amino acid sequence as set out in SEQ ID NO: 4. The pMHC-binding portion may comprise a TCR α variable domain comprising an amino acid sequence having at least 70%, at least 80%, at least 90%, at least 95%, at least 98% or at least 99% sequence identity to SEQ ID NO: 22, wherein the pMHC-binding portion is capable of binding to NY-ESO-1/MHC; and a TCR β variable domain comprising an amino acid sequence having at least 70%, at least 80%, at least 90%, at least 95%, at least 98% or at least 99% sequence identity to SEQ ID NO: 4 wherein the pMHC-binding portion binds NY-ESO-1/MHC.

The pMHC-binding portion of a receptor of the invention may be formed upon complexation of the two polypeptides.

Hence, the extracellular portion of the first polypeptide may comprise: (a) a TCR CDR1α loop, a TCR CDR2α loop and a TCR CDR3α loop; and/or (b) a TCR α variable domain (e.g. as described above), and the extracellular portion of the second polypeptide may comprise: (a) a CDR1β loop, a CDR2β loop and a CDR3β loop; and/or (b) a TCR β variable domain (e.g. as described above), and complexation of the two variable domains form the pMHC-binding portion in the extracellular portion of the pMHC-binding receptor.

Alternatively, the TCR α and β CDR loops and/or variable domains may be comprised on a single polypeptide chain. A single polypeptide chain comprising both a α variable domain and a β variable domain may also comprise a linker or flexible framework region to allow said α and β domains to orientate relative to each other and the pMHC. In such an embodiment, the heterodimeric pMHC-binding receptor of the invention may comprise: (i) a first polypeptide comprising: (a) an extracellular portion comprising both a TCR α variable domain and a TCR β variable domain as described herein; (b) a transmembrane portion; and/or (c) an intracellular portion, and (ii) a second polypeptide comprising a transmembrane portion and an intracellular portion, and optionally a minimal extracellular portion. Upon dimerisation of the first and second polypeptides, parts of the transmembrane portions and/or intracellular portions of the first and second polypeptides may complex to form the transmembrane portion and/or the intracellular portion of the pMHC-binding receptor.

The pMHC-binding portion may be derived from an antibody or an antigen binding fragment thereof, i.e. the pMHC-binding portion of a receptor of the invention may comprise the antigen binding portion of an antibody or an antigen binding fragment thereof (e.g. see FIG. 12). The antibody binding fragment may be a scFv, Fab, a Fab′, a F(ab′)2 fragment, a heavy chain variable domain (VH) or a nanobody (VHH or VNAR).

The pMHC-binding portion may comprise a heavy chain CDR1, CDR2 and CDR3 and light chain CDR1, CDR2 and CDR3. The pMHC-binding portion may additionally comprise antibody light chain framework regions FR1, FR2, FR3 and FR4 and heavy chain framework regions FR1, FR2, FR3 and FR4.

As described herein, the pMHC-binding portion of a receptor of the invention may be formed upon complexation of the two polypeptides. Hence, the extracellular portion of the first polypeptide may comprise: (a) a heavy chain CDR1, CDR2 and CDR3; and/or (b) a heavy chain variable domain of an antibody or an antigen-binding fragment thereof, and the extracellular portion of the second polypeptide may comprise: (a) light chain CDR1, CDR2 and CDR3; and/or (b) a light chain variable domain of the antibody or an antigen-binding fragment thereof, and complexation of the variable domains form the pMHC-binding portion in the extracellular portion of the pMHC-binding receptor.

Alternatively, said heavy chain regions and light chain regions may be comprised on a single polypeptide chain. A single polypeptide chain comprising both light and heavy chain regions may also comprise a linker or flexible framework region to allow said light and heavy chain regions to orientate relative to each other and the pMHC. In such an embodiment, the heterodimeric pMHC-binding receptor of the invention may comprise: (i) a first polypeptide comprising: (a) an extracellular portion comprising both light and heavy chain regions of an antibody as described herein; (b) a transmembrane portion; and/or (c) an intracellular portion, and (ii) a second polypeptide comprising a transmembrane portion and an intracellular portion, and optionally a minimal extracellular portion. Upon dimerisation of the first and second polypeptides, parts of the transmembrane portions and/or intracellular portions of the first and second polypeptides may complex to form the transmembrane portion and/or the intracellular portion of the pMHC-binding receptor.

The extracellular portion may additionally comprise a structural portion which acts as a scaffold to present the pMHC-binding portion in an appropriate structure and orientation for binding to the pMHC. The structural portion is typically formed upon complexation of certain parts of the two polypeptides of the heterodimeric receptor of the invention. The structural portion is typically at the membrane-proximal to the pMHC-binding portion.

The size of the structural portion may be modified to optimise the height of the pMHC-binding portion from the membrane and/or to alter the orientation of the pMHC-binding portion relative to a target antigen. The size of the structural portion may be modified to optimise receptor signalling and/or antigen sensitivity.

The structural portion may be derived from a T cell receptor (e.g. see FIGS. 1B and 12).

For example, the structural portion may comprise a truncated TCR α constant region and a truncated TCR β constant region, such that the structural portion does not mediate CD3 complexation.

The truncated TCR α constant region may not comprise the alpha chain connecting peptide motif (α-CPM). In some embodiments the α-CPM has sequence SEQ ID NO: 45. For example, the truncated TCR α constant region may not comprise an amino acid sequence as set out in SEQ ID NO: 45 or a functionally equivalent sequence variant thereof. Said exclusion of the α-CPM from receptors of the invention may be achieved by means which will be apparent to the skilled person, for example by complete omission or replacement of the CPM or by non-conservative mutation of key residues such as the CXXC motif (SEQ ID NO: 66) such that the receptor of the invention is surface-presented.

The truncated TCR β constant region may not comprise the beta chain connecting peptide motif (β-CPM). In some embodiments the β-CPM comprises sequence SEQ ID NO: 46. The β-CPM may have sequence SEQ ID NO: 43 which is the β-CPM sequence of T cell receptor beta constant variant 1 (TRBC1) (UniProt P01850), or the β-CPM may have sequence SEQ ID NO: 44 which is the β-CPM sequence of TRCB2 (UniProt A0A5B9). For example, the truncated TCR β constant region may not comprise an amino acid sequence as set out in SEQ ID NO: 46 or a functionally equivalent sequence variant thereof. In some embodiments the truncated TCR β constant region may not comprise an amino acid sequence as set out in SEQ ID NO: 43 or 44. Said exclusion of the j-CPM from receptors of the invention may be achieved by means which will be apparent to the skilled person, for example by complete omission or replacement of the CPM or by non-conservative mutation of key residues such as the CXXC motif (SEQ ID NO: 66) such that the receptor of the invention is surface-presented.

The truncated TCR α constant region may not comprise SEQ ID NO: 38 or a functionally equivalent sequence variant thereof.

The truncated TCR β constant region may not comprise SEQ ID NO: 39 or a functionally equivalent sequence variant thereof.

The truncated TCR α constant region may consist of the residues corresponding to N-terminal residues 1 to 94 of the amino acid sequence of a TCR α constant region, for example residues corresponding to N-terminal residues 1 to 94 of amino acid sequence SEQ ID NO: 2.

The truncated TCR β constant region may consist of the residues corresponding to N-terminal residues 1 to 130 of the amino acid sequence of a β constant regions, for example residues corresponding to N-terminal residues 1 to 130 of amino acid sequence SEQ ID NO: 5.

The truncated TCR α constant regions may comprise or consist of an amino acid sequence having ≥70%, ≥80%, ≥90%, ≥least 95% or 100% sequence identity to SEQ ID NO: 6.

In the embodiments where the structural portion is formed upon complexation of two polypeptides, the extracellular portion of the first polypeptide may comprise a truncated TCR α constant region (e.g SEQ ID NO: 6), and the extracellular portion of the second polypeptide may comprise a truncated TCR β constant region (e.g. SEQ ID NO: 9), and complexation of the constant regions form the structural portion in the extracellular portion of the pMHC-binding receptor.

The truncated TCR β constant regions may comprise or consist of an amino acid sequence having ≥70%, ≥80%, ≥90%, ≥least 95% or 100% sequence identity to SEQ ID NO: 9.

The structural portion may be derived from an antibody (e.g. see FIG. 12). For example, the structural portion may be the constant domains of an antibody, such as a light chain constant domain or one of the heavy chain constant domains. The antibody light chain may be kappa or lambda and the heavy chain may be from IgG1, IgG2, IgG3 or IgG4. The antibody heavy chain may be from IgG1. The heavy chain constant domain may be CH1, CH2 or CH3. The heavy chain constant domain may be CH1.

The structural portion may comprise any molecule or fragment thereof which acts as a scaffold and does not compromise binding and/or signalling of the receptor of the invention. Many such molecules or fragments thereof will be apparent to the skilled person. The structural portion may be the same size as, or a substantially similar size to, a full-length TCR constant domain. The structural portion may be compatible with heterodimerisation. In some embodiments, the structural portion may comprise or consist of a mucin-like sequence. Mucin like sequences are characterised by being rich in proline serine and threonine residues, and the serine and threonine residues are heavily O-glycosylated. The mucin-like sequence may be a mucin-like sequence of the extracellular portion of CD43. Mucin-like sequences from other proteins may also be used, such as MUC1, MUC3A, MUC3B, MUC4, MUC12, MUC13, MUC15, MUC16, MUC17, MUC18, MUC20, MUC21, PSGL-1. Stalks of surface proteins that are mucin-like, such as CD8 and CD28, may also be used. Alternatively, the structural portion may comprise or consist of one or more folded domains, or a fragment of the CD28 or CD8 hinge. Alternatively, the structural portion may comprise or consist of one or more folded domains which may be one or more domains having an immunoglobulin fold, such as an immunoglobulin constant domain, or FNIII.

In the embodiments where the structural portion is formed upon complexation of two polypeptides, the extracellular portion of the first polypeptide may comprise the heavy chain constant domain of an antibody (e.g. IgG1-CH1 domain, such as SEQ ID NO: 36), and the extracellular portion of the second polypeptide may comprise the light chain constant domain of the antibody (e.g. SEQ ID NO: 37), and complexation of the constant domains form the membrane proximal structural portion in the extracellular portion of the pMHC-binding receptor.

In some embodiments, the extracellular portion of the pMHC-binding receptor may comprise TCR α and β variable and truncated TCR α and β constant regions, as described herein. In such an embodiment, the pMHC-binding receptor may comprise a first polypeptide comprising TCR α variable and truncated a constant regions, and a second polypeptide comprising TCR β variable and truncated R constant regions. The variable and truncated constant regions may be derived from the same TCR.

In some embodiments, the extracellular portion of the pMHC-binding receptor may comprise antibody light chain and heavy chain variable and constant domains. In such an embodiment, the pMHC-binding receptor may comprise a first polypeptide comprising the heavy chain variable and constant domains of an antibody, and a second polypeptide comprising the light chain variable and constant domains of the antibody. The variable and constant domains may be derived from the same TCR.

In some embodiments, the extracellular portion of the pMHC-binding receptor may comprise antibody light chain and heavy chain variable domains and TCR α and β constant regions. In such an embodiment, the pMHC-binding receptor may comprise a first polypeptide comprising an antibody heavy chain variable domain and a truncated TCR α constant region, and a second polypeptide comprising an antibody light chain variable domain and a truncated TCR β constant region. It is of course within the scope of the invention to pair the antibody heavy variable domain with the truncated TCR β constant region, and vice versa.

In some embodiments, the extracellular portion of the pMHC-binding receptor may comprise the TCR α and β variable domains and antibody light chain and heavy chain constant domains. In such an embodiment, the pMHC-binding receptor may comprise a first polypeptide comprising a TCR α variable domain and an antibody heavy chain constant domain, and a second polypeptide comprising a TCR β variable domain and an antibody light chain constant domain. It is of course within the scope of the invention to pair the TCR α variable domain with the antibody light chain constant domain, and vice versa.

### Transmembrane Portion

Any transmembrane sequences can be used in a pMHC-binding receptor of the invention. However, to minimise CD3 complexation, the transmembrane portion of a receptor of the invention may not comprise the highly conserved charged residues in the α and R chain transmembrane regions (SEQ ID NOs: 47 and 48 respectively). In some embodiments the transmembrane portion of a receptor of the invention may not comprise a TCR α chain transmembrane region or a TCR β chain transmembrane region and may instead comprise a transmembrane region derived from another molecule as described herein. Thus the first polypeptide may not comprise a sequence SEQ ID NO: 47, which is the transmembrane sequence of the T cell receptor alpha constant (TRAC, UniProt P01848). Thus the second polypeptide may not comprise a sequence SEQ ID NO: 48, which is the transmembrane sequence common to both TRBC1 (UniProt P01850) and TRCB2 (UniProt A0A5B9). In some embodiments the second polypeptide may not comprise a sequence SEQ ID NO: 69 which is the transmembrane sequence of TRBC1, or may not comprise a sequence SEQ ID NO: 70 which is the transmembrane sequence of TRBC2.

The transmembrane portion may derive from a CD28, CD8α, CD4 or CD247 molecule (e.g. human CD28, CD8α, CD4 or CD247 molecules). The transmembrane portion may comprise the transmembrane portion of CD28 (e.g. SEQ ID NO: 7), CD8α (e.g. SEQ ID NO: 23), CD4 (e.g. SEQ ID NO: 24) or CD247 (e.g. SEQ ID NO: 25). Hence, the transmembrane portion of the first and/or second polypeptide that form the heterodimer receptor of the invention may comprise any of these transmembrane portions. Typically, the first and second polypeptides comprise the same transmembrane portions.

### Intracellular Portion

The intracellular portion of a pMHC-binding receptor of the invention comprises a signalling portion.

The signalling portion may comprise an immunoreceptor tyrosine-based activation motif (ITAM). An ITAM typically comprises consensus sequence YxxL/Ix₍₆₋₈₎YxxL/I (SEQ ID NO: 40) wherein ‘x’ is any amino acid.

The signalling portion may comprise a pair of ITAMs, e.g. wherein one ITAM is on each of the two polypeptide chains that form the heterodimeric receptor of the invention. The signalling portion may comprise two pairs of ITAMs or three pairs of ITAMs, e.g. wherein two or three ITAMs are on each of the two polypeptide chains that form the heterodimeric receptor of the invention.

The signalling portion may comprise a hemITAM (SEQ ID NO: 41). The signalling portion may comprise one or more hemITAMs.

The signalling portion may comprise the signalling portion of a TCRζ (CD247) (e.g. SEQ ID NO: 8), a CD3ε (SEQ ID NO: 11), a CD3δ (SEQ ID NO: 12), a CD3γ (SEQ ID NO: 13), a DAP12 (e.g. SEQ ID NP: 27), a FcεRIγ (e.g. SEQ ID NO: 32), a FcγRIIa (e.g. SEQ ID NO: 33) or a CD79 molecule (e.g. SEQ ID NO: 34 or 35), e.g. a human TCRζ, a human CD3ε, a human CD3δ, a human CD3γ, a human DAP12, a human FcεRIγ, a human FcγRIIa or a human CD79 molecule. The signalling portion may comprise synthetic cytoplasmic tails which, for example, may comprise ITAM sequences from any of the above signalling molecules or may comprise different combinations of ITAM sequences from any of the above signalling molecules.

Thus, the intracellular portion of the first and/or second polypeptide that form the heterodimer receptor of the invention may comprise a signalling portion comprising any of these signalling portions.

The first and second polypeptides may comprise the same signalling portions. For example, both polypeptides may comprise the TCRζ signalling portion (SEQ ID NO: 8). TCRζ is also known as CD247 or TCRzeta, and is also frequently referred to as CD3ζ or CD3zeta in scientific literature. The terms TCRζ, TCRzeta, CD247, CD3ζ, CD3zeta, and ‘zeta chain’ are used interchangeably herein.

The first and the second polypeptides may comprise different signalling portions. For example, the first polypeptide may comprise a CD3ε signalling portion and the second polypeptide may comprise a CD3δ signalling portion.

The intracellular portion may additionally comprise a co-stimulatory domain, which typically is located at the N-terminal to or within the signalling portion. The co-stimulatory domain comprise the co-stimulatory domain of a CD28, a CD2, a CD226, a CD28H, a CD137 (also known as ‘4-1BB’) or a CD278 molecule (e.g. human CD28, a human CD2, a human CD226, a human CD28H, a human CD137 or a human CD278 molecule). Other suitable co-stimulatory domains will be apparent to the skilled person, such as IL-15Rα, CD134, CD27, CDS, ICAM-1, LTA-1 and ICOS.

Thus, the intracellular portion of the first and/or second polypeptide that form the heterodimer receptor of the invention may additional comprise one or more co-stimulatory domains. The one or more costimulatory domains comprises any of CD2 (SEQ ID NO: 22), CD226 (SEQ ID NO: 28), CD28H (SEQ ID NO: 29), CD137 (SEQ ID NO: 30) and/or CD278 (SEQ ID NO: 31) costimulatory domains. The first and second polypeptides may comprise the same or different co-stimulatory domains.

pMHC-Binding Receptors and Properties

In particular, a pMHC-binding receptor of the invention may comprise:
(i) a first polypeptide comprising:
   (a) an extracellular portion comprising a TCR α variable domain, and optionally a truncated TCR α constant region (e.g. SEQ ID NO: 6);
   (b) a transmembrane region from CD28 (e.g. SEQ ID NO: 7), CD8α (e.g. SEQ ID NO: 23), CD4 (e.g. SEQ ID NO: 24) or CD247 (SEQ ID NO: 25); and
   (c) an intracellular region comprising one or more TCRζ (e.g. SEQ ID NO: 8), CD3ε, CD3δ, CD3γ, DAP12, FcεRIγ, FcγRIIa or CD79 signalling domain; and
(ii) a second polypeptide comprising:
   (a) an extracellular region comprising a TCR β variable domain, and optionally a truncated TCR β constant region (e.g. SEQ ID NO: 11);
   (b) a transmembrane region from CD28 (e.g. SEQ ID NO: 7), CD8α (e.g. SEQ ID NO: 23), CD4 (e.g. SEQ ID NO: 24) or CD247 (SEQ ID NO: 25); and
   (c) an intracellular region comprising one or more TCRζ (e.g. SEQ ID NO: 8), CD3ε, CD3δ, CD3γ, DAP12, FcεRIγ, FcγRIIa or CD79 signalling domain,
optionally wherein the intracellular region of each of the first and the second polypeptides further comprises any of CD2 (SEQ ID NO: 26), CD226 (SEQ ID NO: 28), CD28H (SEQ ID NO: 29), and/or CD137 (SEQ ID NO: 30) costimulatory domains.

For example, a pMHC-binding receptor of the invention may comprise:
(i) a first polypeptide comprising:
   (a) an extracellular portion comprising a TCR α variable domain, and optionally a truncated TCR α constant region (e.g. SEQ ID NO: 6);
   (b) a transmembrane region from CD28 (e.g. SEQ ID NO: 7) or CD8α (SEQ ID NO: 23); and
   (c) an intracellular region comprising a signalling domain from TCRζ (e.g. SEQ ID NO: 8); and
(ii) a second polypeptide comprising:
   (a) an extracellular region comprising a TCR β variable domain and a truncated TCR β constant region (e.g. SEQ ID NO: 9);
   (b) a transmembrane region from CD28 (e.g. SEQ ID NO: 7) or CD8α (SEQ ID NO: 23); and
   (c) an intracellular region comprising a signalling domain from TCRζ (e.g. SEQ ID NO: 8),
optionally wherein the intracellular region of each of the first and the second polypeptides further comprises any of CD2 (SEQ ID NO: 26), CD226 (SEQ ID NO: 28), CD28H (SEQ ID NO: 29), and/or CD137 (SEQ ID NO: 30) costimulatory domains.

The first and/or second polypeptide may additionally comprise a GSG linker at the C-terminal end of the truncated a or p constant region.

For example, a pMHC-binding receptor of the invention may comprise:
(i) a first polypeptide comprising or consisting of an amino acid sequence having ≥70%, ≥80%, ≥90%, ≥95%, ≥96%, ≥97%, ≥98%, ≥99%, or 100% identity to SEQ ID NO: 56; and/or
(ii) a second polypeptide comprising or consisting of an amino acid sequence having ≥70%, ≥80%, ≥90%, ≥95%, ≥96%, ≥97%, ≥98%, ≥99%, or 100% identity to SEQ ID NO: 57.

For example, a pMHC-binding receptor of the invention may comprise:
(i) a first polypeptide comprising or consisting of an amino acid sequence having ≥70%, ≥80%, ≥90%, ≥95%, ≥96%, ≥97%, ≥98%, ≥99%, or 100% identity to SEQ ID NO: 58; and/or
(ii) a second polypeptide comprising or consisting of an amino acid sequence having ≥70%, ≥80%, ≥90%, ≥95%, ≥96%, ≥97%, ≥98%, ≥99%, or 100% identity to SEQ ID NO: 59.

For example, a pMHC-binding receptor of the invention may comprise:
(i) a first polypeptide comprising or consisting of an amino acid sequence having ≥70%, ≥80%, ≥90%, ≥95%, ≥96%, ≥97%, ≥98%, ≥99%, or 100% identity to SEQ ID NO: 60; and/or
(ii) a second polypeptide comprising or consisting of an amino acid sequence having ≥70%, ≥80%, ≥90%, ≥95%, ≥96%, ≥97%, ≥98%, ≥99%, or 100% identity to SEQ ID NO: 57.

For example, a pMHC-binding receptor of the invention may comprise:
(i) a first polypeptide comprising or consisting of an amino acid sequence having ≥70%, ≥80%, ≥90%, ≥95%, ≥96%, ≥97%, ≥98%, ≥99%, or 100% identity to SEQ ID NO: 61; and/or
(ii) a second polypeptide comprising or consisting of an amino acid sequence having ≥70%, ≥80%, ≥90%, ≥95%, ≥96%, ≥97%, ≥98%, ≥99%, or 100% identity to SEQ ID NO: 57.

For example, a pMHC-binding receptor of the invention may comprise:
(i) a first polypeptide comprising or consisting of an amino acid sequence having ≥70%, ≥80%, ≥90%, ≥95%, ≥96%, ≥97%, ≥98%, ≥99%, or 100% identity to SEQ ID NO: 62; and/or
(ii) a second polypeptide comprising or consisting of an amino acid sequence having ≥70%, ≥80%, ≥90%, ≥95%, ≥96%, ≥97%, ≥98%, ≥99%, or 100% identity to SEQ ID NO: 63.

For example, a pMHC-binding receptor of the invention may comprise:
(i) a first polypeptide comprising or consisting of an amino acid sequence having ≥70%, ≥80%, ≥90%, ≥95%, ≥96%, ≥97%, ≥98%, ≥99%, or 100% identity to SEQ ID NO: 64; and/or
(ii) a second polypeptide comprising or consisting of an amino acid sequence having ≥70%, ≥80%, ≥90%, ≥95%, ≥96%, ≥97%, ≥98%, ≥99%, or 100% identity to SEQ ID NO: 65.

Receptors of the invention have minimal interaction, e.g. are unable to complex, with the endogenous CD3 complex.

The CD3 complex comprises a CD3ε:CD3δ heterodimer, a CD3γ:CD3ε heterodimer, and a TCRζ:TCRζ homodimer, each of which associate with canonical TCRs. Nevertheless, receptors of the invention express as a heterodimeric cell surface protein, allowing pMHC-binding and effective signalling. Surprisingly, the inventors have found that the heterodimer, surface-expressed receptors of the invention which lack the capacity to complex with CD3 display enhanced discrimination for their target pMHC, i.e. cross-reactivity with lower affinity, ‘off-target’ pMHCs is reduced as compared to a canonical TCR with the same target binding specificity. By ‘TCR with the same target binding specificity’ it is meant a TCR which binds to the same pMHC as the receptor in question. For example, where the receptor comprises TCR-derived CDR3α and CDR3β loops (these loops having the most residues which contact the peptide antigen), said loops may be also present in the TCR with the same binding specificity. Where the receptor comprises TCR-derived α variable and β variable domains, said domains, or a sequence exhibiting at least 70%, 80%, 90%, 95%, or 98% sequence identity to said domains, may also be present in the TCR having the same target binding specificity. Nevertheless, despite their enhanced antigen discrimination, sensitivity of receptors of the invention to the higher-affinity target antigen is not compromised relative to the TCR having the same target binding specificity.

### Nucleic Acids, Vectors and Host Cells

Also provided is one or more isolated nucleic acids encoding a pMHC-binding receptor of the invention. In some cases, the nucleic acid is collectively present on more than one nucleic acids, but collectively together they are able to encode a pMHC-binding receptor of the invention.

However, in some embodiments the pMHC-binding receptor is encoded by a single polynucleotide. In some such cases, the single polynucleotide is expressed as a fusion protein. In some such cases, the fusion protein is cleaved post-translationally into the first and second polypeptide chains of the pMHC-binding receptors of the invention. Endopeptidases suitable for cleavage of the fusion protein into its first and second polypeptide chains are well known in the art.

Accordingly, the polynucleotide of the invention may encode an endopeptidase cleavage site located between the nucleotide sequences encoding the first and second polypeptide chains, such that the fusion protein comprises an endopeptidase cleavage site located between the first and second polypeptide chains. Alternatively, the single polynucleotide may encode a self-cleaving peptide, such as a 2A self-cleaving peptide, located between the nucleotide sequences encoding the first and second polypeptide chains. 2A self-cleaving peptides induce ribosomal skipping during their translation, such that a peptide bond is not formed. Thus, a 2A peptide located between the first and second polypeptide chains results in translation of a first and a second polypeptide chain. In some embodiments, the 2A peptide is a P2A peptide. In some embodiments the P2A is located between the first and second polypeptide chains and C-terminal to the first polypeptide chain and N-terminal to the second polypeptide chain (e.g. see Examples and SEQ ID NOs: 54 and 55).

Nucleic acids which encode a pMHC-binding receptor of the invention can be obtained by methods well known to those skilled in the art. For example, DNA sequences coding for part or all of the antibody heavy and light chains may be synthesised as desired from the corresponding amino acid sequences.

The nucleic acid may be a DNA sequence. The nucleic acid may be an RNA sequence, such as mRNA. A vector may comprise the nucleic acid.

The vector may be a viral vector. Conventional viral based expression systems could include retroviral, alpha-retroviral, lentivirus, adenoviral, adeno-associated (AAV) and herpes simplex virus (HSV) vectors for gene transfer. Non-viral transduction vectors include transposon based systems including PiggyBac and Sleeping Beauty systems. Methods for producing and purifying such vectors are known in the art.

The vector may be a cloning vector or an expression vector. A suitable vector may be any vector which is capable of carrying a sufficient amount of genetic information, and allowing expression of a polypeptide of the invention.

The vector is preferably an RNA vector. Suitable RNA vectors include the RNA vectors as described in Schutsky, Keith, et al., Oncotarget 6.30 (2015): 28911 and Beatty, Gregory L., et al., Gastroenterology 155.1 (2018): 29-32.

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

A nucleic acid may be provided in the form of an expression construct (or ‘expression cassette’), which includes control sequences operably linked to the inserted sequence, thus allowing for expression of a pMHC-binding receptor of the invention in vivo. Hence, also provided is one or more expression cassettes encoding the one or more nucleic acids that encode a pMHC-binding receptor of the invention. These expression cassettes, in turn, are typically provided within vectors (e.g. plasmids or recombinant viral vectors). Hence, also provided is a vector encoding a pMHC-binding receptor of the invention. Further provided are vectors which collectively encode a pMHC-binding receptor of the invention.

The vector may be a human artificial chromosome. Human artificial chromosomes are described in e.g. Kazuki et al., Mol. Ther. 19(9): 1591-1601 (2011), and Kouprina et al., Expert Opinion on Drug Delivery 11(4): 517-535 (2014).

The vector may be a non-viral delivery system, such as DNA plasmids, naked nucleic acid (e.g. naked RNA), and nucleic acid complexed with a delivery vehicle, such as a liposome.

The nucleic acids, expression cassettes or vectors described herein may be introduced into a host cell, e.g. by transfection. Hence, also provided is a host cell comprising the one or more nucleic acids, expression cassettes or vectors of the invention. The nucleic acids, expression cassettes or vectors described herein may be introduced transiently or permanently into the host cell, allowing expression of an antibody from the one or more nucleic acids, expression cassettes or vectors. Such host cells include transient, or preferably stable higher eukaryotic cell lines, such as mammalian cells or insect cells, lower eukaryotic cells, such as yeast, or prokaryotic cells, such as bacteria cells. Particular examples of cells include mammalian HEK293, such as HEK293F, HEK293T, HEK293S or HEK Expi293F, CHO, HeLa, NS0 and COS cells, or any other cell line used herein.

Typically, the host cell is an immune effector cell of the invention. The nucleic acids, expression cassettes or vectors described herein may be introduced transiently into the host cell.

Also provided is a cell comprising one or more of the invention, one or more expression constructs of the invention, or one or more vectors of the invention. Also provided is a cell comprising a pMHC-binding receptor of the invention. The cell may be an immune cell, optionally an immune effector cell, for example a T lymphocyte, optionally an inflammatory T lymphocyte, a cytotoxic T lymphocyte, a regulatory T lymphocyte, or a helper T lymphocyte. T lymphocyte may be a CD8+ cytotoxic T lymphocyte.

The polynucleotide or vector of the invention may be an mRNA for administering to patients, e.g. mRNA vaccination. The patient's T cells may then express the receptor of the invention and/or the accessory receptor of the invention in vivo. Such mRNA molecules and associated methods are described in reference 5.

Also provided is a kit suitable for transforming and/or transfecting an immune effector cell or a population of immune effector cells to generate an immune effector cell or population of immune effector cells of the invention. The kit comprises a nucleic acid or vector described herein. The kit may comprise further agents such as those discussed herein that improve transfection or transformation efficacy.

### Methods

The invention also relates to a method of identifying a pMHC-binding receptor specific to a target pMHC of interest, wherein the method comprises screening a library of pMHC-binding receptors of the invention for high affinity binding to said target pMHC.

Also provided is a method of identifying low-affinity or ‘off-target’ binding interactions comprising screening a library of MHC-presented peptides (pMHCs) for binding to a pMHC-binding receptor of the invention, optionally wherein said library does not comprise the pMHC-binding receptor's target pMHC.

Also provided is a method of assessing discrimination capacity of a pMHC-binding receptor of the invention comprising (a) exposing the pMHC-binding receptor to its high affinity target pMHC (i.e. the pMHC for which it is specific) and determining the concentration of pMHC required for signalling; (b) exposing the pMHC-binding receptor to one or more low affinity pMHCs and determining the concentration of these pMHCs required for signalling; (c) comparing the concentration of high affinity pMHC against the concentration of low affinity pMHC required to induce comparable signalling of cells expressing the pMHC-binding receptor, wherein a large difference in concentration indicates good discrimination. Weak or absent signalling induced by low affinity pMHC where the high affinity target pMHC induces signalling also indicates good discrimination.

Signalling may be measured, for example, by induction of a molecule downstream in the pMHC-binding receptor's expression cascade, for example a cytokine, for example but not limited to IL-2, or a surface molecule, for example but not limited to CD69, or target cell killing, for example but not limited to LDH release.

### Pharmaceutical Composition

Also provided is a composition comprising an immune effector cell or population of immune effector cells of the invention. The immune effector cell or population of immune effector cells may be at least 1% of the total cells in the composition, such as at least 5%, at least 10%, at least 15 at least 20%, at least 25%, at least 30%, at least 35%, at least 40%, at least 45%, at least 50%, at least 60%, at least 70%, at least 80%, at least 90%, at least 95%, at least 96%, at least 97%, at least 98%, at least 99% or at least 99.9% of the total cells in the composition. The total cells in the composition may consist or consist essentially of the immune effector cell or population of immune effector cells of the invention, i.e. no other cells are detectable in the composition.

The composition may be a pharmaceutical composition. The pharmaceutical composition may comprise a pharmaceutically acceptable carrier. Suitable pharmaceutically acceptable carriers comprise aqueous carriers, diluents or excipients. Examples of suitable carriers include all aqueous and non-aqueous isotonic sterile injection solutions which may contain anti-oxidants, buffers and solutes, which render the composition isotonic with the blood of the intended recipient; aqueous and non-aqueous sterile suspensions, which may include suspending agents and thickening agents, dispersion media, antifungal and antibacterial agents, isotonic and absorption agents and the like. It will be understood that compositions of the invention may also include other supplementary physiologically active agents.

The carrier is typically pharmaceutically “acceptable” in the sense of being compatible with the other ingredients in the composition and not injurious to the subject. Compositions include those suitable for parenteral administration, including subcutaneous, intramuscular, intravenous and intradermal administration. The compositions may conveniently be presented in unit dosage form and may be prepared by any method well known in the art of pharmacy. Such methods include preparing the carrier for association with the isolated T cells. In general, the compositions are prepared by uniformly and intimately bringing into association any active ingredients with liquid carriers.

The composition may be suitable for parenteral administration. In another embodiment, the composition is suitable for intravenous administration. Compositions suitable for parenteral administration include aqueous and non-aqueous isotonic sterile injection solutions which may contain anti-oxidants, buffers, bactericides and solutes, which render the composition isotonic with the blood of the intended recipient; and aqueous and non-aqueous sterile suspensions which may include suspending agents and thickening agents.

The composition described herein may be prepared in a manner known in the art and are those suitable for parenteral administration to mammals, particularly humans, comprising a therapeutically effective amount of the composition with one or more pharmaceutically acceptable carriers or diluents. The composition may comprise at least about 1×10⁶ to about 1×10¹² of the immune effector cells of the invention.

The present disclosure also contemplates the combination of the composition described herein with other active agents and/or in addition to other treatment regimens or modalities such as radiation therapy or surgery. When the composition described herein is used in combination with known active agents, the combination may be administered either in sequence (either continuously or broken up by periods of no treatment) or concurrently or as an admixture.

Suitable anti-cancer agents will be known to persons skilled in the art.

Treatment in combination is also contemplated to encompass the treatment with either the composition of the invention followed by a known treatment, or treatment with a known agent followed by treatment with the composition of the invention, for example, as maintenance therapy.

For example, in the treatment of cancer it is contemplated that the composition of the present invention may be administered in combination with an alkylating agent (such as mechlorethamine, cyclophosphamide, chlorambucil, ifosfamidecysplatin, or platinum-containing alkylating agents such as cisplatin, carboplatin and oxaliplain), and anti-metabolite (such as a purine or pyrimidine analogue or an anti-folate agent, such as azathioprine and mercaptopurine), an anthracycline (such as daunorubicin, doxorubicin, epirubicin idarubicin, valrubicin, mitoxantrone or anthracycline analog), a plant alkaloid (such as a vinca alkaloid or a taxane, such as vincristine, vinblastine, vinorelbine, vindesine, paclitaxel or doestaxel), a topoisomerase inhibitor (such as a type I or type II topoisomerase inhibitor), a podophyllotoxin (such as etoposide or teniposide), a tyrosine kinase inhibitor (such as imatinib mesylate, nilotinib or dasatinib), an adenosine receptor inhibitor (such as A2aR inhibitors, SCH58261, CPI-444, SYN115, ZM241385, FSPTP or A2BR inhibitors such as PSB-1115), adenosine receptor agonists (such as CCPA, IB-MECA and CI-IB-MECA), a checkpoint inhibitor, including those of the PDL-1:PD-1 axis, nivolumab, pembrolizumab, atezolizumab, BMS-936559, MEDI4736, MPDL33280A or MSB0010718C), an inhibitor of the CTLA-4 pathway (such as ipilimumab and tremelimumab), an inhibitor of the TIM-3 pathway or an agonist monoclonal antibody that is known to promote T cell function (including anti-OX40, such as MEDI6469; and anti-4-BB, such as PF-05082566).

The invention also provides a kit or article of manufacture including a pharmaceutical composition as described above.

The invention also provides a kit for use in a therapeutic application mentioned above, the kit comprising: (a) a container holding a polypeptide, nucleic acid, vector or pharmaceutical composition of the invention; and (b) a label or package insert with instructions for use.

Suitable containers include, for example, bottles, vials, syringes, blister pack, etc. The containers may be formed from a variety of materials such as glass or plastic. The container holds a therapeutic composition which is effective for treating the condition and may have a sterile access port (e.g, the container may be an intravenous solution bag or a vial having a stopper pierceable by a hypodermic injection needle). The label or package insert indicates that the therapeutic composition is used for treating the condition of choice. In an embodiment, the label or package insert includes instructions for use and indicates that the therapeutic or prophylactic composition can be used to treat a cancer or other condition described herein.

The kit may further comprise a further container comprising a pharmaceutically-acceptable buffer, such as bacteriostatic water for injection (BWFI), phosphate-buffered saline, Ringer's solution and dextrose solution. It may further comprise other materials desirable from a commercial and user standpoint, which would be known to persons skilled in the art, suitable examples of which include other buffers, diluents, filters, needles, and syringes.

### Therapeutic Uses

Also described herein is use of a pMHC-binding receptor, polynucleotide(s), vector(s), expression cassette(s), immune effector cell or population of immune effector cells, or a pharmaceutical composition of the invention, in a method of treatment of the human or animal body by therapy, e.g. for use as a medicament.

For instance, also provided is a method of treating cancer in a subject, the method comprising administering to the subject an effective amount of an immune effector cell or a population of immune effector cells of the invention. Hence, the invention also provides pMHC-binding receptor, polynucleotide(s), vector(s), expression cassette(s), immune effector cell or population of immune effector cells, or a pharmaceutical composition of the invention for use in a method of treating cancer. The invention also provides the use of a pMHC-binding receptor, polynucleotide(s), vector(s), expression cassette(s), immune effector cell or population of immune effector cells of the invention for the manufacture of a medicament for the treatment of cancer. The invention also provides the use of a pMHC-binding receptor, polynucleotide(s), vector(s), expression cassette(s), immune effector cell or population of immune effector cells, or a pharmaceutical composition of the invention to treat cancer. The cancer may be any cancer, such as a solid cancer. The cancer may be a malignancy listed in Table 1. The cancer may be haematological malignancy or B cell cancer.

Also provided is a method of treating or preventing an infection in a subject, the method comprising administering to the subject an effective amount of an immune effector cell or a population of immune effector cells of the invention. Hence, the invention also provides pMHC-binding receptor, polynucleotide(s), vector(s), expression cassette(s), immune effector cell or population of immune effector cells, or a pharmaceutical composition of the invention for use in a method of treating or preventing an infection.

Also provided is a method of treating or preventing an inflammatory disease in a subject, the method comprising administering to the subject an effective amount of an immune effector cell or a population of immune effector cells of the invention. Hence, the invention also provides pMHC-binding receptor, polynucleotide(s), vector(s), expression cassette(s), immune effector cell or population of immune effector cells, or a pharmaceutical composition of the invention for use in a method of treating or preventing an inflammatory disease.

The invention also provides the use of a pMHC-binding receptor, polynucleotide(s), vector(s), expression cassette(s), immune effector cell or population of immune effector cells, or a pharmaceutical composition of the invention for the manufacture of a medicament for the treatment or prevention of an infection.

The invention also provides the use of a pMHC-binding receptor, polynucleotide(s), vector(s), expression cassette(s), immune effector cell or population of immune effector cells, or a pharmaceutical composition of the invention to treat or prevent an infection. The infection may be a chronic infection.

The invention also provides the use of a pMHC-binding receptor, polynucleotide(s), vector(s), expression cassette(s), immune effector cell or population of immune effector cells, or a pharmaceutical composition of the invention for the manufacture of a medicament for the treatment or prevention of an inflammatory disease.

The invention also provides the use of a pMHC-binding receptor, polynucleotide(s), vector(s), expression cassette(s), immune effector cell or population of immune effector cells, or a pharmaceutical composition of the invention to treat or prevent an inflammatory disease. The inflammatory disease may be an autoimmune disease.

Also provided is a method of performing adoptive cell therapy in a subject, the method comprising administering to the subject an effective amount of an immune effector cell or a population of immune effector cells of the invention.

Hence, the invention also provides a pMHC-binding receptor, polynucleotide(s), vector(s), expression cassette(s), immune effector cell or population of immune effector cells of the invention, or a pharmaceutical composition for use in adoptive cell therapy. The invention also provides the use of a pMHC-binding receptor, polynucleotide(s), vector(s), expression cassette(s), immune effector cell or population of immune effector cells, or a pharmaceutical composition of the invention for the manufacture of a medicament for adoptive cell therapy.

The invention also provides the use of a pMHC-binding receptor, polynucleotide(s), vector(s), expression cassette(s), immune effector cell or population of immune effector cells, or a pharmaceutical composition of the invention adoptive cell therapy.

The therapeutic uses and methods may comprise administering a therapeutically effective amount of the immune effector cell or population of immune effector cells.

Also provided is a method of formulating a composition for treating cancer, wherein said method comprises mixing an immune effector cell or population of immune effector cells of the invention with an acceptable carrier to prepare said composition.

The subject may have been previously treated for the cancer, such as using adoptive cell therapy.

The therapeutic methods and uses may comprise, prior to treatment with an immune effector cell or population of immune effector cells of the invention, determining whether the cancer expresses a target antigen specifically targeted by immune effector cell or population of immune effector cells of the invention.

The method may comprise selecting an immune effector cell or population of immune effector cells based on the expression of the target antigen by the cancer, so that the immune effector cell or population of immune effector cell is specific for the cancer. The method may comprise transfecting or transforming an immune effector cell with a nucleic acid of the invention in response to information on the expression of the target antigen by the cancer.

The therapeutic methods and uses described herein may comprise inhibiting the disease state (i.e. the cancer), for example by arresting its development and/or causing regression of the disease state until a desired end point is reached. The therapeutic methods and uses of the invention may comprise achieving a partial response, a full response by the cancer. The therapeutic methods and uses of the invention may achieve remission of the cancer.

The therapeutic methods and uses described herein may delay the growth of the cancer, arrest the growth of the cancer and/or reverse the growth of the cancer. The therapeutic methods and uses of the invention may reduce the size of the cancer by at least 10%, such as at least 20%, at least 30%, at least 40%, at least 50%, at least 60%, at least 70%, at least 80%, at least 90% or by 100%.

Typically, the therapeutic methods and uses are for a human subject in need thereof. However, non-humans animals such as non-human mammals are also contemplated. The non-human mammals may be rats, rabbits, sheep, pigs, cows, cats or dogs.

The dose of the immune effector cell or population of immune effector cells may vary depending on the age and size of a subject, as well as on the disease, conditions and route of administration. The immune effector cell or population of immune effector cells may be administered at a dose of about 1×10⁶ to about 1×10¹² cells. The immune effector cell or population of immune effector cells may be administered at a dose of about 1×10⁵ cells/kg to about 1×10¹¹ cells/kg body weight.

The immune effector cell or population of immune effector cells may be administered as a single dose. The immune effector cell or population of immune effector cells may be administered in a multiple dose regimen. For example, the initial dose may be followed by administration of a second or plurality of subsequent doses. The second and subsequent doses may be separated by an appropriate time. For example, the doses between doses may be administered once about every week, once about every 2 weeks, once about every 3 weeks, once about every four weeks, or once about every month.

The immune effector cell or population of immune effector cells may be administered intravenously.

The immune effector cell or population of immune effector cells may be administered with one or more additional therapy, such as one or more additional therapeutic agents. The additional therapeutic agent may be an anti-tumour agent. The additional therapeutic may be an additional immune effector cell.

Combined administration of the immune effector cell or population with the additional therapeutic agent may be achieved in a number of different ways. All the components may be administered together in a single composition. Each component may be administered separately as part of a combined therapy.

For example, the immune effector cell or the population of immune effector cells of the invention may be administered before, after or concurrently with the additional therapeutic agent. The additional therapy may be chemotherapy, radiotherapy and/or surgery.

Prior to administration of the immune effector cell or population of immune effector cells of the invention, the subject may undergo lymphodepletion. Lymphodepletion may be achieved via administration to the subject with fluradabine, cyclophosphamide and/or bendamustine. Lymphodepletion may be carried out for at least about one day, such as about 2 days or about 3 days.

The biological activity and/or therapeutic efficacy of the administered immune effector cell or population of immune effector cells may be measured by known methods. For example, the method may comprise imaging, such as magnetic resonance imaging.

TABLE 1
Examples of antigen-associated malignancies which may be
addressed by the pMHC-binding receptors of the invention.
pMHC-binding receptors of the invention may bind the peptides
from said antigens presented on any MHC molecule, including
MHC-I or MHC-II and any allele variants thereof.

| Antigen | Associated Malignancy |
|---|---|
| α-Folate receptor | Ovarian cancer, epithelial cancers |
| CAIX | Renal-cell carcinoma |
| CD19 | B cell malignancies, Refractory |
| | Follicular, Lymphoma, ALL, CLL |
| CD20 | Lymphomas, B cell malignancies, B-cell |
| | lymphomas, Mantle cell lymphoma, |
| | indolent B cell lymphomas |
| CD22 | B cell malignancies |
| CD23 | CLL |
| CD24 | Pancreatic adenocarcinoma |
| CD30 | Lymphomas, Hodgkin lymphoma |
| CD33 | AML |
| CD38 | Non Hodgkin lymphoma |
| CD44v7/8 | Cervical carcinoma |
| CEA | Colorectal cancer |
| EGFRvIII | Glioblastoma |
| EGP-2 | Multiple malignancies |
| EGP-40 | Colorectal cancer |
| EphA2 | Glioblastoma |
| erb-B2 | Breast cancer, Prostate cancer, |
| | Colon cancer, Various tumors |
| erb-B 2,3,4 | Breast cancer |
| FBP | Ovarian cancer |
| Fetal acethylcholine | Rhabdomyosarcoma |
| e receptor | |
| G_{D2} | Neuroblastoma, Melanoma, |
| | Ewing sarcoma |
| G_{D3} | Melanoma |
| Her-2 | Medulloblastoma, Pancreatic |
| | adenocarcinoma, Glioblastoma, |
| | Osteosarcoma, Ovarian cancer |
| HMW-MAA | Melanoma |
| IL-11Rα | Osteosarcoma |
| IL-13R-α2 | Glioma, Glioblastoma, Medulloblastoma |
| KDR | Tumor neovasculature |
| κ-light chain | B-cell malignancies (B-NHL, CLL) |
| Lewis Y | Various carcinomas, Epithelial |
| | derived tumors |
| L1-cell adhesion | Neuroblastoma |
| molecule | |
| MAGE-A1 | Melanoma |
| Mesothelin | Mesothelioma |
| Murine CMV infected | Murine CMV |
| cells | |
| MUC1 | Breast, Ovary |
| MUC16 | Ovary |
| NKG2D Ligands | Myeloma, ovarian & other tumors |
| NY-ESO-1 | Melanoma, Multiple myeloma |
| Oncofetal antigen | Various tumors |
| (h5T4) | |
| PSCA | Prostate carcinoma |
| scFv-TCRζ | |
| PSMA | Prostate cancer/tumor |
| | vasculature |
| Prostate/tumor | scFv-CD28-TCRζ |
| vasculature | |
| ROR1 | B-CLL and mantle cell lymphoma |
| Targeting via | Various tumors |
| mAb IgE | |
| TAG-72 | Adenocarcinomas |
| TAX | Adult T-cell leukemia/lymphoma (ATL) |
| VEGF-R2 | Tumor neovasculature |
| Biotinylated | Various tumors-ovarian |
| molecules | |

### Embodiments of the Invention

1. A MHC-presented peptide (pMHC)-binding receptor comprising two polypeptides forming a heterodimer which comprises: (a) an extracellular portion comprising a pMHC-binding portion; (b) a transmembrane portion; and (c) an intracellular portion comprising a signalling domain.
2. The pMHC-binding receptor of embodiment 1, wherein:
   (i) the first polypeptide comprises: (a) an extracellular portion; (b) a transmembrane portion; (c) an intracellular portion; and
   (ii) the second polypeptide comprises: (a) an extracellular portion; (b) a transmembrane portion; (c) an intracellular portion.
3. The pMHC-binding receptor of embodiment 1 or embodiment 2, wherein the pMHC-binding portion is derived from a T cell receptor.
5. The pMHC-binding receptor of embodiment 1 or 2, wherein the pMHC-binding portion is derived from an antibody or an antigen-binding fragment thereof.
6. The pMHC-binding receptor of embodiment 5, wherein the extracellular portion of the first polypeptide comprises the heavy chain variable domain of an antibody or an antigen-binding fragment thereof, the extracellular portion of the second polypeptide comprises the light chain variable domain of the antibody or an antigen-binding fragment thereof, and complexation of the variable domains form the pMHC-binding portion in the extracellular portion of the pMHC-binding receptor.
7. The pMHC-binding receptor of any one of embodiments 1 to 6, wherein the extracellular portion of the pMHC-binding receptor further comprises a structural portion which is membrane proximal to the pMHC-binding portion and is formed upon complexation of the two polypeptides.
8. The pMHC-binding receptor of embodiment 7, wherein the structural portion is derived from a T cell receptor.
9. The pMHC-binding receptor of embodiment 8, wherein the extracellular portion of the first polypeptide comprises a truncated TCR α constant region (e.g SEQ ID NO: 6), and the extracellular portion of the second polypeptide comprises a truncated TCR β constant region (e.g. SEQ ID NO: 9), and complexation of the constant regions form the membrane proximal structural portion in the extracellular portion of the pMHC-binding receptor.
10. The pMHC-binding receptor of embodiment 7, wherein the structural portion is derived from an antibody.
11. The pMHC-binding receptor of embodiment 10, wherein the extracellular portion of the first polypeptide comprises a heavy chain constant domain of an antibody (e.g. IgG1-CH1 domain, such as SEQ ID NO: 36), and the extracellular portion of the second polypeptide comprises the light chain constant domain of the antibody (e.g. SEQ ID NO: 37), and complexation of the constant domains form the membrane proximal structural portion in the extracellular portion of the pMHC-binding receptor.
12. The pMHC-binding receptor of any one of embodiments 1 to 11, wherein the transmembrane portion of the first and/or second polypeptide comprises the transmembrane portion of CD28 (e.g. SEQ ID NO: 7) or CD8α (e.g. SEQ ID NO: 23).
13. The pMHC-binding receptor of any one of embodiments 1 to 12, wherein the intracellular portion of the first and/or second polypeptide comprises a signalling domain comprising one or more pairs of immunoreceptor tyrosine-based activation motifs (ITAMs) (e.g. SEQ ID NO: 40) or one or more hemITAMs (e.g. SEQ ID NO: 41).
14. The pMHC-binding receptor of any one of embodiments 1 to 13, wherein the intracellular portion of the first and/or second polypeptide comprises the intracellular portion of one or more TCRζ chains (e.g. SEQ ID NO: 8).
15. The pMHC-binding receptor of any one of embodiments 1 to 14, wherein the intracellular portion of the first and/or second polypeptide additionally comprises one or more co-stimulatory domains, optionally wherein the one or more costimulatory domains comprises any of CD2 (SEQ ID NO: 26), CD226 (SEQ ID NO: 28), CD28H (SEQ ID NO: 29), and/or CD137 (SEQ ID NO: 30) costimulatory domains.
16. The pMHC-binding receptor of any one of embodiments 1 to 15, wherein the pMHC-binding receptor does not associate with a CD3 complex.
17. The pMHC-binding receptor of any one of embodiments 1 to 16, wherein
   (i) the first polypeptide comprises: (a) an extracellular portion comprising a TCR α variable domain and a truncated TCR α constant region (e.g. SEQ ID NO: 6); (b) a transmembrane region from CD28 (e.g. SEQ ID NO: 7) or CD8α (SEQ ID NO: 23); and (c) an intracellular region comprising a signalling domain from TCRζ (e.g. SEQ ID NO: 8); and
   (ii) the second polypeptide comprises: (a) an extracellular region comprising a TCR β variable domain and a truncated TCR β constant region (e.g. SEQ ID NO: 9); (b) a transmembrane region from CD28 (e.g. SEQ ID NO: 7) or CD8α (SEQ ID NO: 23); and (c) an intracellular region comprising a signalling domain from TCRζ (e.g. SEQ ID NO: 8),
   optionally wherein the intracellular region of each of the first and the second polypeptides further comprises any of CD2 (SEQ ID NO: 26), CD226 (SEQ ID NO: 28), CD28H (SEQ ID NO: 29), and/or CD137 (SEQ ID NO: 30) costimulatory domains.
18. One or more isolated polynucleotides encoding the first polypeptide and/or second polypeptide of the pMHC-binding receptor as defined in any one of embodiments 1 to 17.
19. One or more expression constructs comprising one or more polynucleotides as defined in embodiment 18, or one or more vectors comprising the one or more polypeptides of embodiment 18 or the one or more expression constructs.
20. A cell comprising one or more polynucleotides as defined in embodiment 18, one or more expression constructs or vectors as defined in embodiment 19.
21. A cell comprising a pMHC-binding receptor as defined in any one of embodiments 1 to 17, optionally wherein the cell is an immune cell, such as an immune effector cell (e.g. a CD8+ cytotoxic T lymphocyte).
22. A pharmaceutical composition comprising: (i) one or more polynucleotides as defined in embodiment 18, one or more expression constructs or vectors as defined in embodiment 19, or a cell as defined in embodiment 20 or 21, and (ii) a pharmaceutically acceptable carrier.
23. A pMHC-binding receptor as defined in any one of embodiments 1 to 17, one or more polynucleotides as defined in embodiment 18, one or more expression constructs or vectors as defined in embodiment 18, or a cell as defined in embodiment 20 or 21, or a pharmaceutical composition as defined in embodiment 22 for use as a medicament, such as for use in treating cancer, in treating an infection, optionally a chronic infection, or in treating an inflammatory disease, optionally an autoimmune disease.
24. A method, such as an ex vivo method, of preparing a population of immune cells for adoptive cell therapy, the method comprises culturing the cell of embodiment 20 or 21 to produce a population of immune cells.
25. A population of immune cells produced by the method of embodiment 24.

### Other

It is to be understood that different applications of the disclosed pMHC-binding receptors, immune effector cells, or pharmaceutical compositions of the invention may be tailored to the specific needs in the art. It is also to be understood that the terminology used herein is for the purpose of describing particular embodiments of the invention only, and is not intended to be limiting.

A ‘TCR-based EDR’ or ‘EDR’ is a pMHC-binding receptor in which the extracellular pMHC-binding portion is derived from a TCR. An ‘antibody-based EDR’ is a pMHC-binding receptor in which the extracellular pMHC-binding portion derived from an antibody.

In addition as used in this specification and the appended claims, the singular forms “a”, “an”, and “the” include plural references unless the content clearly dictates otherwise. Thus, for example, reference to “a pMHC-binding receptor” includes two or more “pMHC-binding receptors”.

Furthermore, when referring to “≥x” herein, this means equal to or greater than x. When referred to “≤x” herein, this means less than or equal to x.

For the purpose of this invention, in order to determine the percent identity of two sequences (such as two polynucleotide or two polypeptide sequences), the sequences are aligned for optimal comparison purposes (e.g. gaps can be introduced in a first sequence for optimal alignment with a second sequence). The nucleotide or amino acid residues at each position are then compared. When a position in the first sequence is occupied by the same nucleotide or amino acid as the corresponding position in the second sequence, then the nucleotides or amino acids are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence is 95% identical to SEQ ID NO: 3, SEQ ID NO: 3 would be the reference sequence. To assess whether a sequence is at least 95% identical to SEQ ID NO: 3 (an example of a reference sequence), the skilled person would carry out an alignment over the length of SEQ ID NO: 3, and identify how many positions in the test sequence were identical to those of SEQ ID NO: 3. If at least 95% of the positions are identical, the test sequence is at least 95% identical to SEQ ID NO: 3. If the sequence is shorter than SEQ ID NO: 3, the gaps or missing positions should be considered to be non-identical positions.

The skilled person is aware of different computer programs that are available to determine the identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

In some embodiments, the receptors of the invention are humanised. Humanisation is the modification of amino acid residues within non-human derived sequences to reduce immunogenicity when administered to a human subject. Humanisation may be by grafting of non-human functional sequences (e.g. CDRs) onto human structural or scaffolding sequences, but is now more commonly achieved by mutation of residues within a non-human sequence to a counterpart residue occupying the equivalent position in a corresponding human homolog (‘back-mutation’). Preferably only the structural regions are humanised such that binding capabilities are not affected.

Unless otherwise provided, sequences of domains, chains, regions and motifs of proteins are sequences from human homologs of said proteins.

All publications, patents and patent applications cited herein, whether supra or infra, are hereby incorporated by reference in their entirety.

The following examples illustrate the invention.

### Example 1—Engineering Enhanced Discrimination Receptors

The inventors engineered various pMHC-binding receptors having enhanced pMHC-discrimination capability, referred to herein as enhanced discrimination receptors (EDRs).

EDRs do not Associate with the CD3 Complex and are Surface-Expressed

TCRα-β-Jurkat cells were lentivirally transduced with either the 1G4 TCR or a 1G4 EDR. Both the IG4 TCR and 1G4 EDR are specific for the NY-ESO-1/HLA-A*02 pMHC complex.

The IG4 EDR was expressed as a single fusion polypeptide and cleaved upon translation into two polypeptides using the P2A self-cleaving peptide (SEQ ID NO: 3). The two polypeptides formed a heterodimeric complex (adopting a structure as shown in FIG. 1i), with an antigen binding domain at the membrane distal portion which mimics the antigen binding domain of the TCR.

PE-Tetramers of the 9V pMHC antigen were used to detect surface levels of each antigen receptor and UCHT antibody to detect surface CD3ε levels.

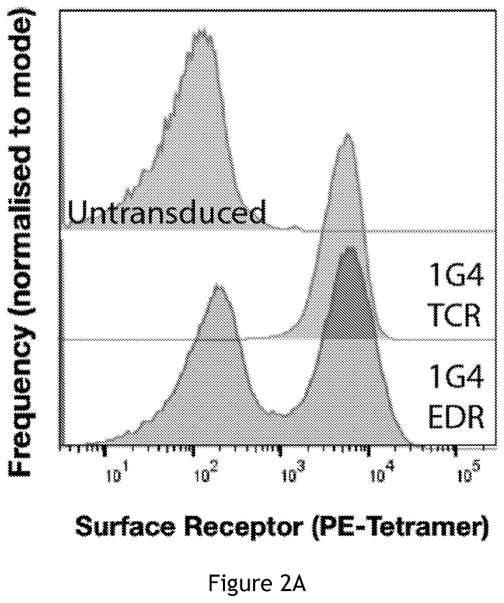

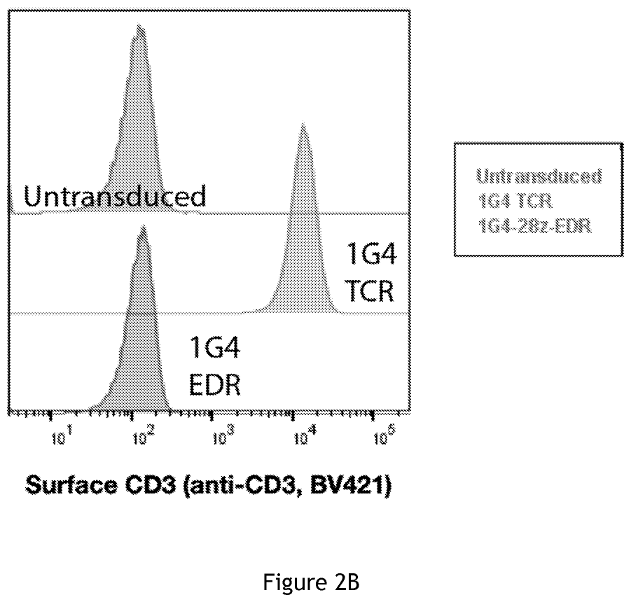

As shown in FIG. 2, the 1G4 EDR was detected at the cell surface using pMHIC tetramers without CD3ε upregulation, indicating these receptors are CD3-independent, which is not the case for the 1G4 TCR. Replacement of the α and β transmembrane helices within the EDR and associated loss of the highly conserved charged residues comprised therein which mediate interaction of a αβTCR with the CD36F and CD3γε heterodimers and TCRζζ homodimer was expected to abrogate EDR-CD3 association. Nevertheless, EDRs were surface-expressed, likely due to the absence of the membrane-proximal region of the αβTCR and associated absence of the hydrophobic alpha-chain connecting peptide motif (alpha-CPM).

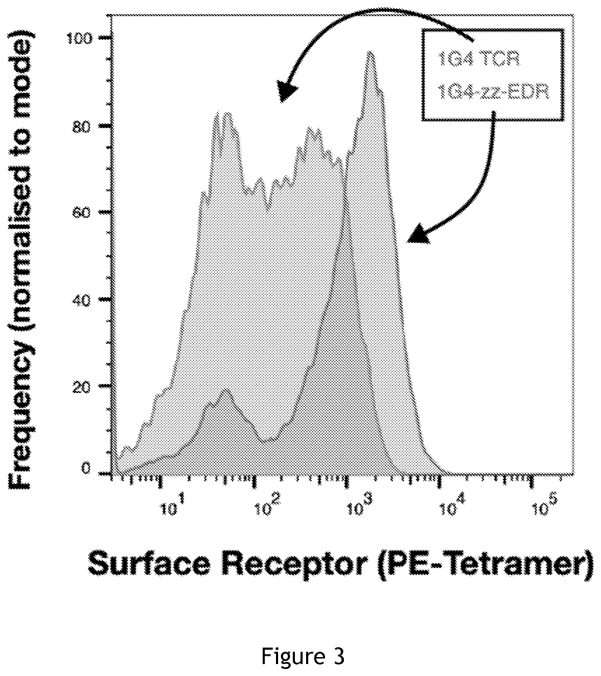

As shown in FIG. 3, surface expression of the 1G4 EDR on primary human CD8+ T cells lentivirally transduced with the 1G4 EDR, as detected with 9V pMHIC antigen, is higher than of 1G4 TCR on CD8+ T cells transduced in the same way with 1G4 TCR. This shows that, unlike the 1G4 TCR, the EDR does not need to compete for CD3 subunits with endogenous TCR.

### Materials and Methods

### Plasmid Constructs

Generation of lentivirus infectious particles was conducted using a third generation system which included: the (i) pMD2.G (Addgene #12259), (ii) pRSV-Rev (Addgene #12253), and (iii) pMDLg/pRRE (Addgene #12251) packaging plasmids. The sequences of diverse lentiviral transfer vectors are detailed in the next sub-section. Said lentiviral transfer vectors encoded receptors that bind the NY-ESO-1/iLA-A*02 pMHIC complex.

1G4 T cell receptor (TCR), comprised of a variable alpha (SEQ ID NO. 1) and constant alpha (SEQ ID NO. 2) domain, a P2A self-cleaving peptide (SEQ ID NO. 3), followed by a variable beta (SEQ ID NO. 4) and constant beta (SEQ ID NO. 5) domain. The variable domains confer specificity.

1G4-z/z-EDR was expressed as a single fusion polypeptide comprising, from N-terminus to C-terminus, a variable alpha domain (SEQ ID NO. 1) followed by a truncated variant of the constant alpha domain (SEQ ID NO. 6), a transmembrane region derived from CD28 (SEQ ID NO. 7), a TCRζ (CD247) signalling tail (SEQ ID NO. 8), a glycine-serine linker (GSG), a P2A self-cleaving peptide (SEQ ID NO. 3), a variable beta domain (SEQ ID NO. 4), a truncated variant of the constant beta domain (SEQ ID NO. 9), a transmembrane region derived from CD28 (SEQ ID NO. 7), and a TCRζ chain (CD247) signalling tail (SEQ ID NO. 8).

1G4-28z-EDR was expressed as a single fusion polypeptide comprising, from N-terminus to C-terminus, a variable alpha domain (SEQ ID NO. 1) followed by a truncated variant of the constant alpha domain (SEQ ID NO. 6), a transmembrane region derived from CD28 (SEQ ID NO. 7), a signalling motif derived from CD28 (SEQ ID NO. 10), a TCRζ chain (CD247) signalling tail (SEQ ID NO. 8), a glycine-serine linker (GSG), a P2A self-cleaving peptide (SEQ ID NO. 3), a variable beta domain (SEQ ID NO. 4), a truncated variant of the constant beta domain (SEQ ID NO. 9), a transmembrane region derived from CD28 (SEQ ID NO. 7), and a TCRζ chain (CD247) signalling tail (SEQ ID NO. 8).

### Production of Lentiviral Supernatants

293T cells (ATCC CRL-3216) were allowed to attach overnight and, the following morning, transfected with a mixture of packaging plasmids (950 ng of pRSV-Rev, 370 ng of pMD2.G, 950 ng of pMDLg/pRRE, and 1000 ng of the corresponding lentiviral transfer plasmid). Transfection was executed using the X-tremeGENE HP Transfection Reagent using a ratio of 3 μL of transfection reagent to 1 g of DNA. 293T cells were then incubated at 37° C. 10% CO2 (v/v) for 48 h, and the lentiviral supernatant filtered through a 0.45 μm cellulose acetate syringe filter. The supernatant was used to transduce either Jurkat or primary CD8+ T cells.

### Cell Lines and Primary Cells

Cell lines used were:
293T cells (ATCC CRL-3216) (referred to herein as “primary human CD8+ T cells”);
Jurkat E6.1 TCR α⁻β⁻ cells, a derivative from the Jurkat E6.1 clone (ATCC TIB-152); and
NALM6 (clone G5, ATCC CRL-3273).

Human leukocyte cones were acquired, with CD8+ T cells being isolated using the RosetteSep CD8+ T cell Enrichment Cocktail (STEMCELL). Briefly, each millilitre of the leukocyte cone was mixed with 150 μL of CD8+ T cell Enrichment Cocktail and incubated at room temperature for 20 min. The mixture was diluted with an equal volume of PBS and layered over Ficoll Paque Plus density gradient media (GE Health Sciences). Samples were centrifuged at 1200*g for 20 min (brake off). Cells were resuspended in RPMI 1640 media supplemented with: 10% FBS, penicillin-streptomycin (100 U/mL and 100 g/mL, respectively), and recombinant human IL-2 at 100 U/mL.

Isolated CD8+T lymphocytes were adjusted to a concentration of 500,000 viable cells per mL and mixed with human anti-CD3/CD28 Dynabeads at a 1:1 ratio of cells to beads. Cells were rested overnight and, the following morning, 1.5 million CD8+ T cells transduced with the corresponding lentiviral supernatant encoding the receptor of interest. Seventy-two hours post-transduction, cells were subjected either to puromycin (pLEX_307 backbone) or G418 (pLEX307-NeoR backbone) selection. For cells subjected to puromycin selection, an initial pulse of 500 ng/mL was first conducted. This was followed, after 48 h, by a 1000 ng/mL pulse of puromycin. Cells subjected to G418 selection were pulsed in a similar manner, except that the initial pulse of G418 corresponded to a concentration of 500 g/mL and the second one, to a concentration of 1000 g/mL of G418. From that point onwards, cells were split by dilution every 2-3 days, and maintained at an approximate concentration of 500,000 cells/mL with IL-2 supplementation (100 U/mL). Regular T cell maintenance continued for 15-17 days post-isolation.

### Example 2—EDRs Exhibit Enhanced Discrimination

### CD69 Assay

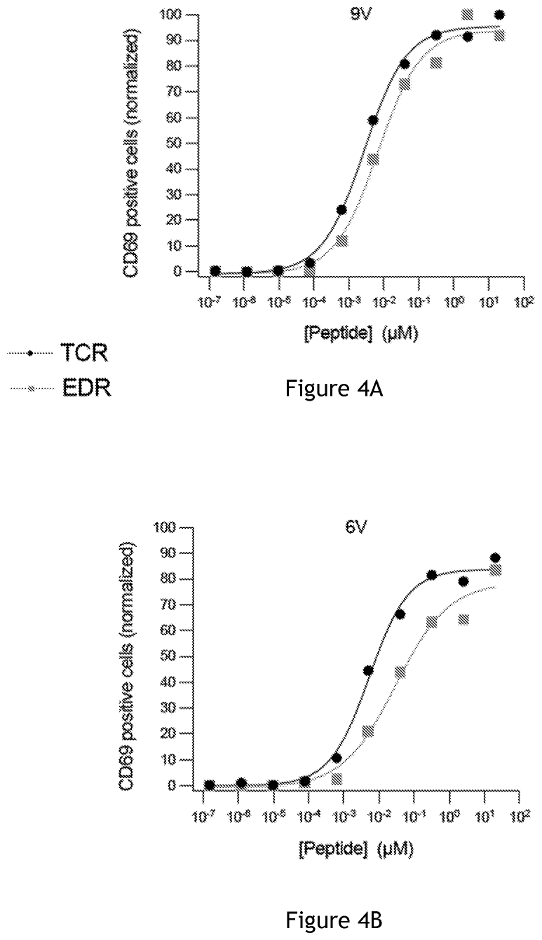

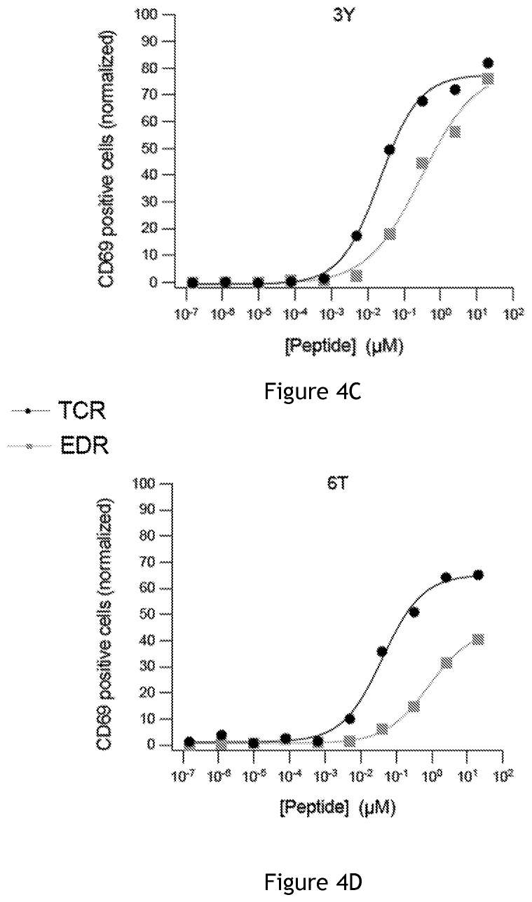

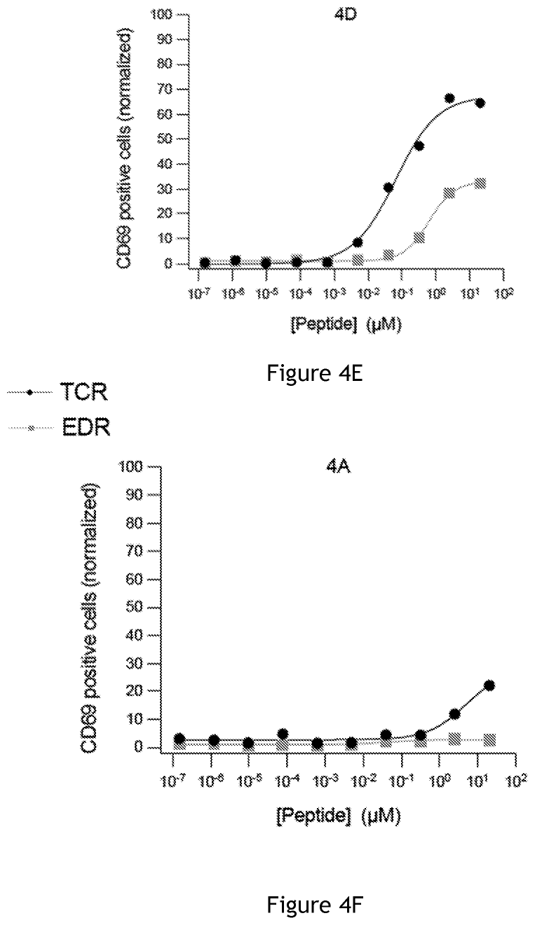

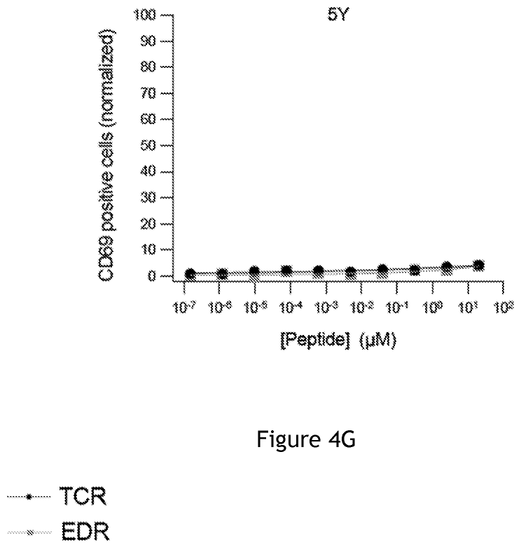

CD69 is an early activation marker of T cells which is rapidly induced after stimulation via the TCR. As such it is a useful reporter of T cell stimulation. When primary human CD8+ T cells transduced with the 1G4 TCR or 1G4-z/z-EDR were stimulated by Nalm6 target cells loaded with different concentrations of peptide antigens (highest concentration was 20 μM) and surface CD69 was measured using flow cytometry after 20 hours, the EDR exhibited enhanced antigen discrimination (FIG. 4).

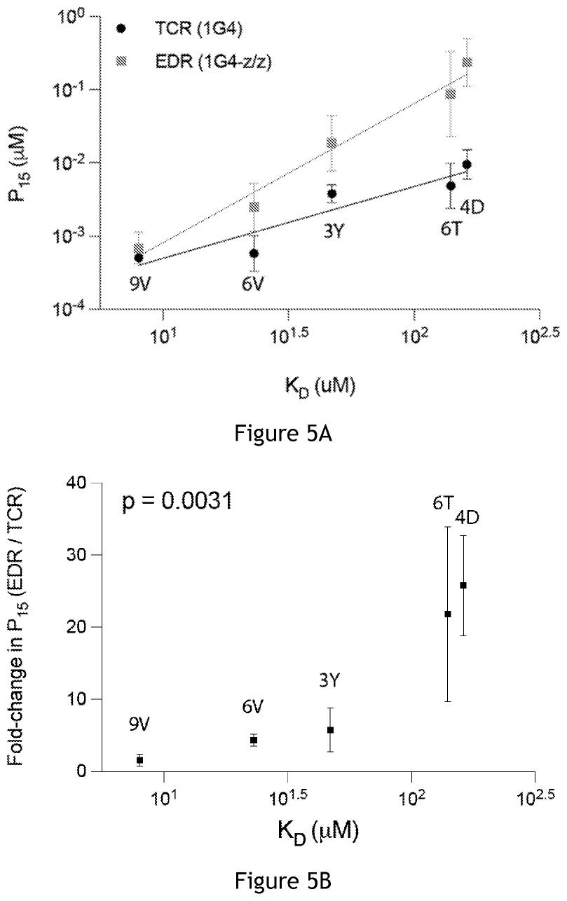

Using the same data to plot the concentration of antigen required to activate 15% of T cells above background (P15) and fold-change in P15 against TCR/pNMC affinity data measured using SPR revealed that the EDR required similar antigen concentration to activate T cells when the antigen affinity was high (see, e.g., peptide 9V of FIG. 5) but required >20-fold more antigen to elicit T cell activation when the antigen affinity was low (e.g. peptides 6T and 4D of FIG. 5).

The EDR thus requires a higher concentration of lower-affinity to induce T cell activation compared to the TCR when measuring surface CD69, but sensitivity to higher-affinity antigens is not compromised. Therefore, the EDR exhibits higher antigen discrimination to the TCR but the same antigen sensitivity as the TCR.

### IL-2 Assay

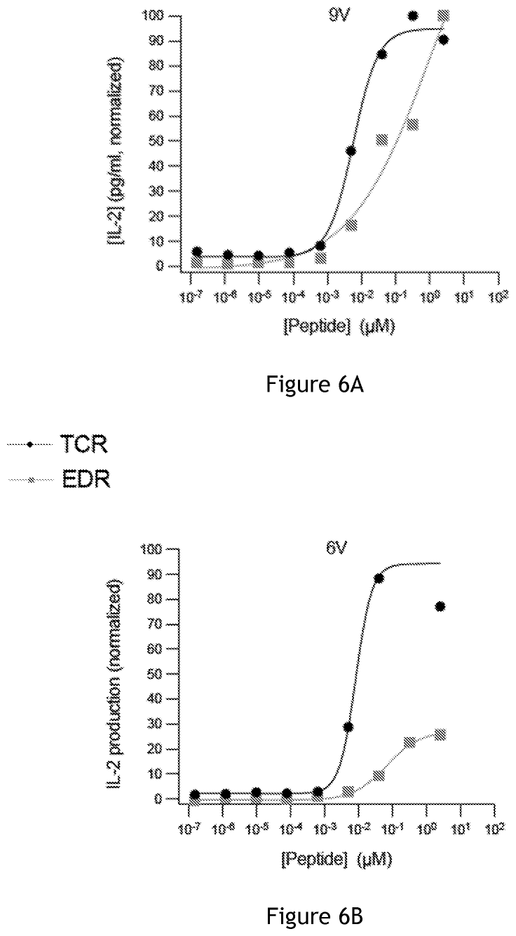

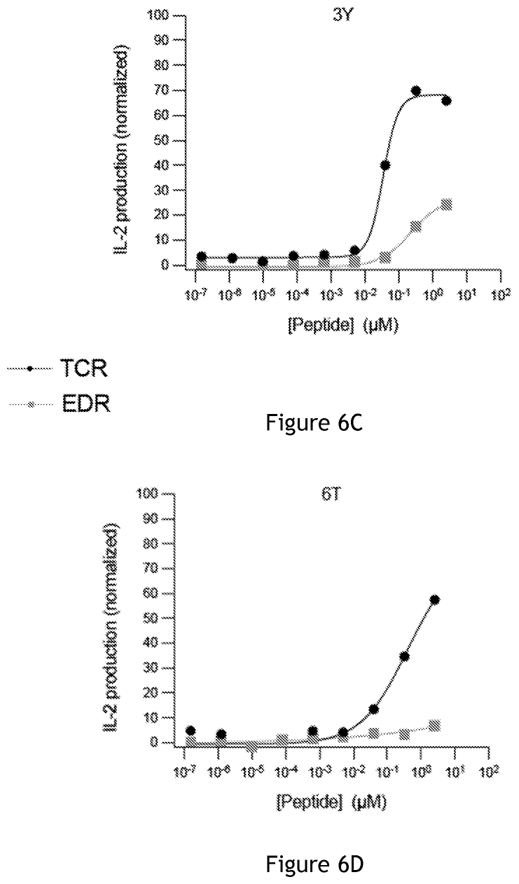

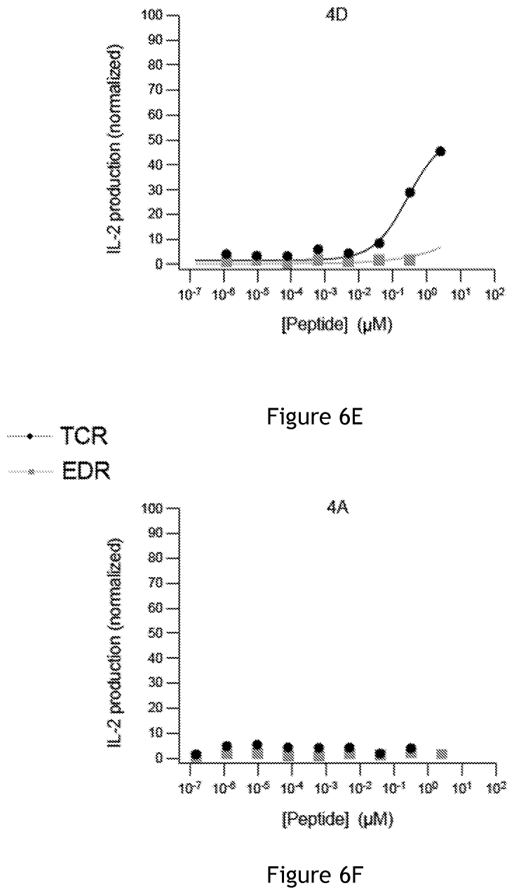

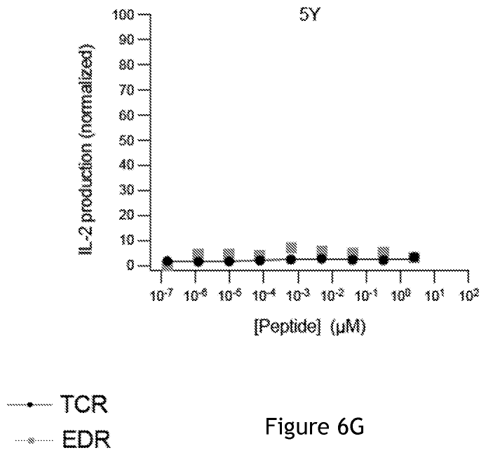

Primary human CD8+ T cells transduced with the 1G4 TCR or 1G4-z/z-EDR were stimulated by Nalm6 target cells loaded with the indicated concentration of peptide antigen and supernatant IL-2 was measured using ELISA after 20 hours. The EDR exhibited effective stimulation for target peptide antigen 9V, but reduced stimulation for off-target antigens (FIG. 6). In other words, the EDR exhibited enhanced antigen discrimination compared to the TCR.

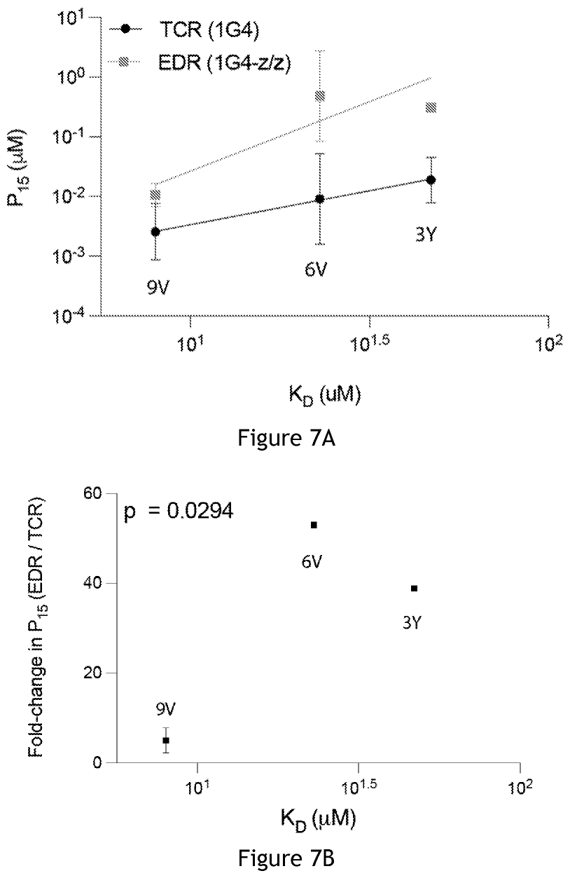

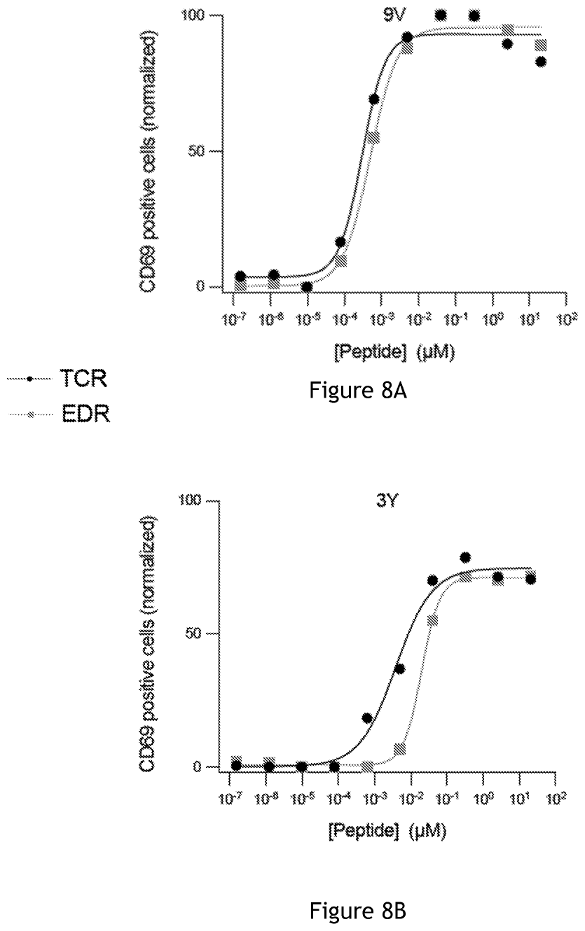

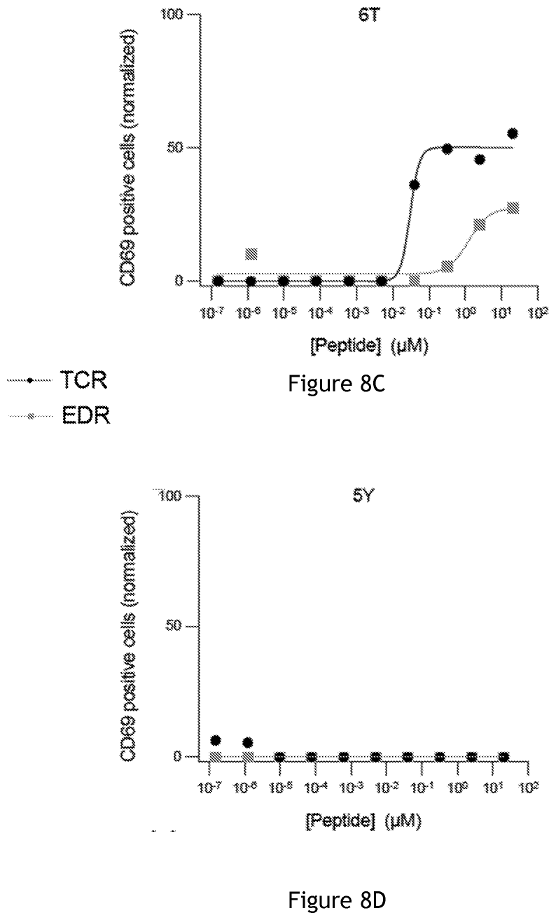

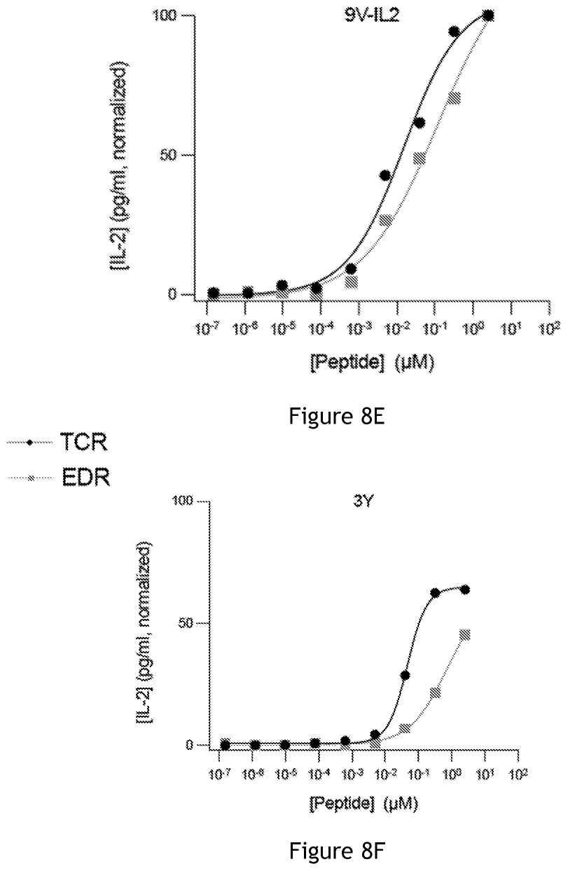

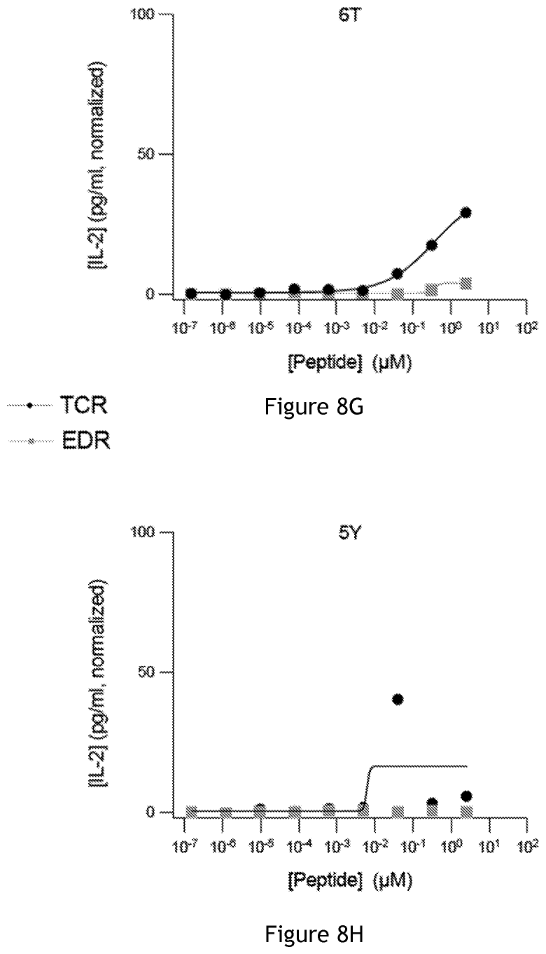

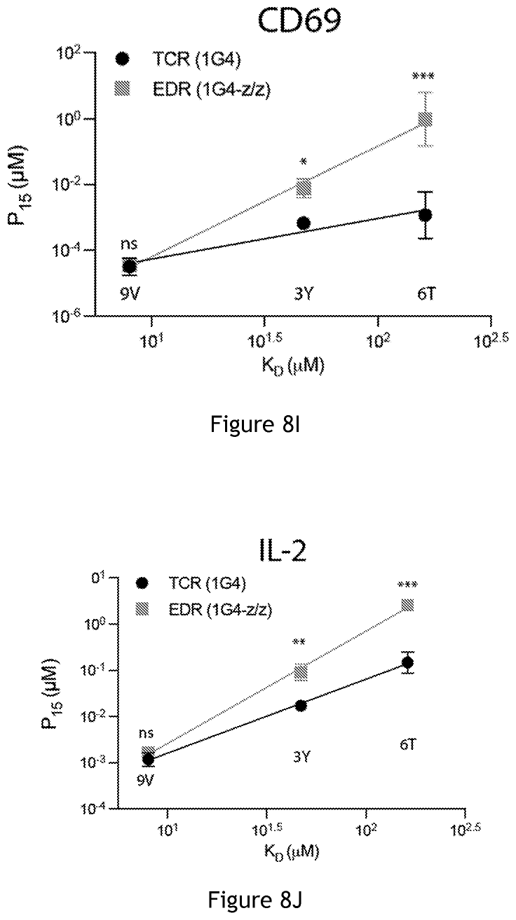

Using the same data to plot the concentration of antigen required to activate 15% of T cells above background (P15) and fold-change in P15 against TCR/pMHC affinity data measured using SPR revealed that the EDR required similar antigen concentration to activate T cells when the antigen affinity was high (see, e.g., peptide 9V of FIG. 7) but required >20-fold more antigen to elicit T cell activation when the antigen affinity was low (e.g. peptides 6V and 3Y of FIG. 7).

The EDR thus required a higher concentration of lower-affinity but not higher-affinity antigens to induce T cell activation compared to the TCR when measuring IL-2 production.

The same CD69 and IL-2 assays were performed using the U87 glioblastoma target cell line. Consistent with the above, the EDR exhibited enhanced antigen discrimination compared to the TCR when measuring CD69 or IL-2 on the U87 glioblastoma cell line.

### CD28 Co-Stimulation

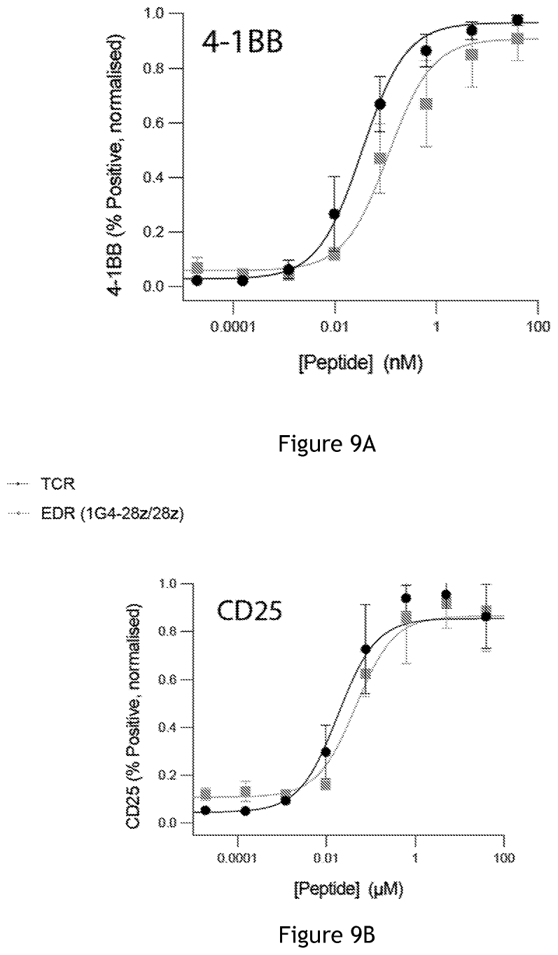

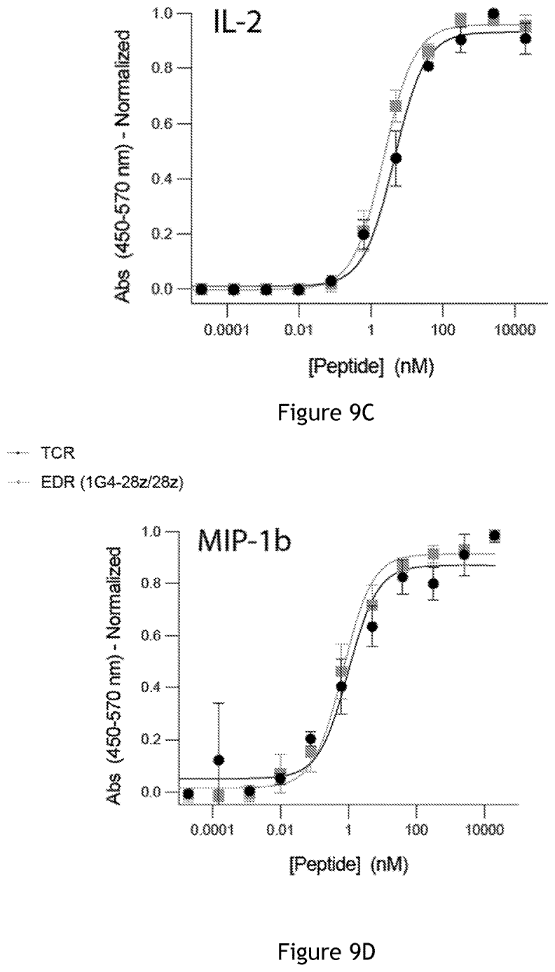

Primary human CD8+ T cells were transduced with the 1G4 TCR or 1G4-28z/28z-EDR and stimulated for 20 hours by T2 target cells loaded with 9V peptide antigen at increasing concentrations. Levels of surface 4-1BB and surface CD25 were measured by flow cytometry (FIGS. 9 A and B) and IL-2 and MIP-1b measured by ELISA (FIGS. 9 C and D). Results demonstrate that an EDR comprising CD28 co-stimulation motifs achieves the same antigen sensitivity as the TCR to a high affinity antigen.

### A6 EDR

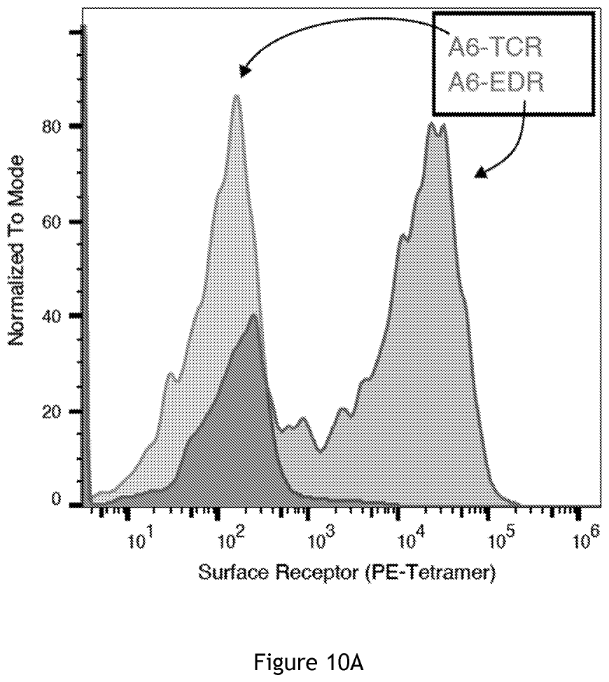

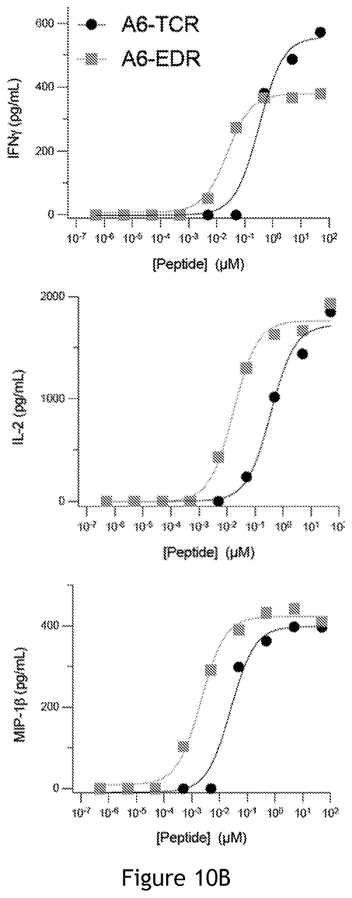

An A6-z/z-EDR that recognises the Tax peptide from HTLV was generated and surface expression of the A6 TCR or the A6 EDR on primary human CD8+ T cells detected with PE-tetramers of the Tax pMHC. As with the 1G4 EDR, surface expression of the A6 is higher than that of the A6 TCR (FIG. 10A).

Cytokine assays of primary human CD8+ T cells transduced with the A6 TCR or A6 EDR and stimulated by U87 glioblastoma target cells loaded with the indicated concentration of the wild-type Tax peptide antigen demonstrated that the A6 EDR is even more sensitive than the A6 TCR for its target antigen.

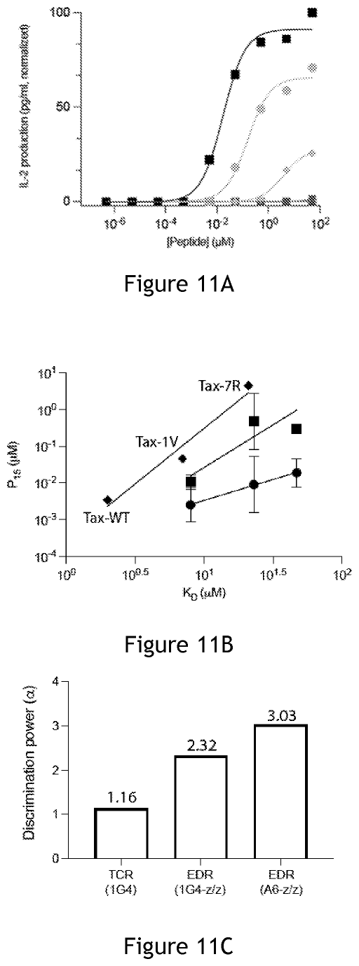

IL-2 cytokine assays of primary human CD8+ T cells transduced by the A6 EDR and stimulated with U87 glioblastoma loaded with target and off-target antigens were conducted and results compared against 1G4 EDR and 1G4 TCR data. The A6 EDR exhibited antigen discrimination capability similar to the 1G4 EDR, and exhibited enhanced discrimination relative to the 1G4 TCR (FIG. 11).

### Materials and Methods

Materials and methods used in this Example were the same as those in Example 1, unless otherwise stated.

### Plasmid Constructs

1G4 TCR, 1G4-z/z-EDR and 1G4-28z/28z-EDR are specific for the NY-ESO-1/HLA-A*02 pMHC complex, and were prepared as described in Example 1.

A6 TCR and A6-z/z-EDR are specific for the Tax/HLA-A*02 pMHC complex.

A6 TCR comprised of a variable alpha domain derived from the A6 TCR (SEQ ID NO. 17), and constant alpha (SEQ ID NO. 2) domain, a P2A self-cleaving peptide (SEQ ID NO. 3), followed by a variable beta domain derived from the A6 TCR (SEQ ID NO. 18), and a constant beta domain (SEQ ID NO. 4). The variable domains confer specificity.

A6-z/z-EDR was expressed as a single fusion polypeptide comprising, a variable alpha domain derived from the A6 TCR (SEQ ID NO. 17), followed by a truncated variant of the constant alpha domain (SEQ ID NO. 6), a transmembrane region derived from CD28 (SEQ ID NO. 7), a TCRζ chain (CD247) signalling tail (SEQ ID NO. 8), a glycine-serine linker (GSG), a P2A self-cleaving peptide (SEQ ID NO. 3), a variable beta domain derived from the A6 TCR (SEQ ID NO. 18), a truncated variant of the constant beta domain (SEQ ID NO. 9), a transmembrane region derived from CD28 (SEQ ID NO. 7), and a TCRζ chain (CD247) signalling tail (SEQ ID NO. 8).

### T Cell Stimulation Assays

Stimulation with suspension cells (NALM6): The NALM6 suspension cell line was used as a surrogate APC, onto which altered peptide ligands (APLs) of the NY-ESO-1 peptide were loaded. Chiefly, 3.3×10⁴ NALM6 cells were seeded in a V-bottom 96-well plate in a volume of 110 μL of full RPMI. Serial dilutions of the corresponding APL were prepared also using full RPMI. 110 μL of the diluted peptide were then added to the NALM6 cell suspension, and the cells incubated for 90 min at 37° C. and 5% CO2 (v/v). The NALM6 cells were then washed with full RPMI and resuspended in 110 μL of this media. A total of 30,000 NALM6 cells (corresponding to 100 μL) were transferred to a U-bottom 96-well plate. After this, a total of 60,000 CD8+ T cells (in a volume of 100 L) were added to each well, establishing a simple 2:1 effector:target ratio. Plates were gently centrifuged (20*g for 3 min) to promote contact between the T cells and the APCs. Co-cultures were conducted during 4 or 5 h, with the cells being incubated at 37° C. and 5% CO2 (v/v).

Stimulation with adherent cells (U87): Thirty-thousand U87 cells, in a volume of 100 μL of DMEM, were seeded in a tissue culture treated flat-bottom 96 well plate and grown overnight. On the following day, additional 100 μL of DMEM containing a given concentration of a given APL was added to these cells. Afterwards, the U87 samples were incubated for 90 min at 37° C. and 5% CO2 (v/v).

Then, the peptide containing media was removed by aspiration and 60,000 CD8+ T cells (in a volume of 200 μL) per well were promptly added to each well. The co-culture was then spun (20*g for 3 min), and incubated at 37° C. and 5% CO2 (v/v). Co-cultures were conducted from APLs of either the NY-ESO-1 or the Tax peptides, and the incubations lasted either 5 or 24 h.

### Flow Cytometry

After an experiment was concluded, cells were transferred to a 96-well plate with a V bottom and centrifuged at 520 g for 5 min at 4° C. Cells were first stained (30 min at RT) with a fixable viability dye (Zombie near-infrared, 1:500 working dilution) in a volume of 50 μL of PBS. Samples were then stained (30 min at 4° C.) with conjugated flow cytometry antibodies, which were previously diluted in 50 L of PBS. Working dilutions ranged from 1:200 for commercial antibodies to 1:500 for pMHC 9V or Tax tetramers. Cells were then washed, and resuspended in PBS with 1% BSA. Sample acquisition was conducted in a BD X-20 or Cytoflex cytometer, with analyses conducted with the FlowJo suite.

Of note, detection of the multiple receptors was conducted with fluorescent pMHC tetramers. The tetramers were composed of biotinylated, refolded 9V or Tax pMHC molecules (a gift from M. Kutuzov) complexed with PE streptavidin. Fluorescent tetramers were prepared by vigorously mixing 66.6 g of monomeric pMHC with step-wise additions of 10 μL of PE streptavidin every 10 min (10 additions of PE streptavidin over 100 min).

### Cytokine Measurements

Following the co-culture experiments, supernatants were assayed for levels of diverse cytokines or chemokines, including but not limited to: IFN-γ, IL-2, and MIP-13. The assays were conducted using commercial human cytokine uncoated ELISA kits provided by Thermo Fisher Scientific. In order to determine the levels of cytokine production, the instructions of the manufacturer were followed.

### Example 3—EDRs

Further useful EDRs are as follows:

1G4 family:
1a. 1G4-z/z-EDR (also abbreviated to “1G4 EDR”);
1b. 1G4-28z/28z-EDR;
1c. 1G4-e/d-EDR;
1d. 1G4-g/e-EDR;
1e. 1G4-z12X/z12X-EDR;
if 1G4-z1XX/z1XX-EDR;
1g. 1G4-zXXX/zXXX-EDR;

A6 family:
2a. A6-z/z-EDR (also “A6 EDR”);
2b. A6-28z/28z-EDR;
2c. A6-e/d-EDR;
2d. A6-g/e-EDR;
2e. A6-z12X/z12X-EDR;
2f. A6-z1XX/z1XX-EDR;
2g. A6-zXXX/zXXX-EDR;

a3a family:
3a. a3a-z/z-EDR;
3b. a3a-28z/28z-EDR;
3c. a3a-e/d-EDR; a3a-g/e-EDR;
3d. a3a-z12X/z12X-EDR;
3e. a3a-z1XX/z1XX-EDR;
3f. a3a-zXXX/zXXX-EDR;

wt/c51 family (derived from the wt/c51 TCR, which is a modified variant of the 1G4 TCR comprising a variable alpha domain having sequence SEQ ID NO: 1 and a variable beta domain having sequence SEQ ID NO. 21)
4a. wt/c51-z/z EDR;
4b. wt/c51-28z/28z-EDR;
4c. wt/c51-e/d-EDR; 4d. wt/c51-g/e-EDR; 4e. wt/c51-z12X/z12X-EDR;
4f. wt/c51-z1XX/z1XX-EDR; 4g. wt/c51-zXXX/zXXX-EDR;

c259/wt family (derived from the c259/wt TCR, which is a modified variant of the 1G4 TCR comprising a variable alpha domain having sequence SEQ ID NO: 22 and a variable beta domain having sequence SEQ ID NO: 4):
5a. c259/wt-z/z-EDR;
5b. c259/wt-28z/28z-EDR;
5c. c259/wt-e/d-EDR;
5d. c259/wt-g/e-EDR;
5e. c259/wt-z12X/z12X-EDR;
5f. c259/wt-z1XX/z1XX-EDR;
5g. c259/wt-zXXX/zXXX-EDR;
   the family of EDRs that are identical to those described in 1a to 6g, except that they use a transmembrane (TM) sequence derived from human CD8α (SEQ ID NO. 23) instead of one derived from human CD28;
   the family of EDRs that are identical to those described in 1a to 6g, except that they use a transmembrane (TM) sequence derived from human CD4 (SEQ ID NO. 24) instead of one derived from human CD28;
   the family of EDRs that are identical to those described in 1a to 6g, except that they use a transmembrane (TM) sequence derived from human CD247 (SEQ ID NO. 25) instead of one derived from human CD28;
   the family of −2z-EDRs, which are identical to those described in 1b, 2b, 3b, 4b, 5b, and 6b except that they use a co-stimulatory sequence derived from human CD2 (SEQ ID NO. 26) instead of one derived from human CD28.
   the family of -DAP12-EDRs, which are identical to those described in 1a, 2a, 3a, 4a, 5a, and 6a except that the first and second instance of the signalling portion of human CD247 is substituted by that of human DAP12 (SEQ ID NO. 27) instead of one derived from human CD247;
   the family of -CD226z-EDRs, which are identical to those described in 1b, 2b, 3b, 4b, 5b, and 6b except that they use a co-stimulatory sequence derived from human CD226 (DNAM-1) (SEQ ID NO. 28) instead of one derived from human CD28;
   the family of -CD28 Hz-EDRs, which are identical to those described in 1b, 2b, 3b, 4b, 5b, and 6b except that they use a co-stimulatory sequence derived from human CD28H (SEQ ID NO. 29) instead of one derived from human CD28;
   the family of -CD137z-EDRs, which are identical to those described in 1b, 2b, 3b, 4b, 5b, and 6b except that they use a co-stimulatory sequence derived from human CD137 (SEQ ID NO. 30) instead of one derived from human CD28;
   the family of -CD278z-EDRs, which are identical to those described in 1b, 2b, 3b, 4b, 5b, and 6b except that they use a co-stimulatory sequence derived from human CD278 (SEQ ID NO. 31) instead of one derived from human CD28;
   the family of -FcεRIγ-EDRs, which are identical to those described in 1a, 2a, 3a, 4a, 5a, and 6a except that the first and second instance of the signalling portion of human CD247 is substituted by that of human FcεRIγ (SEQ ID NO. 32);
   the family of -FcγRIIa-EDRs, which are identical to those described in 1a, 2a, 3a, 4a, 5a, and 6a except that the first and second instance of the signalling portion of human CD247 is substituted by that of human FcγRIIa (SEQ ID NO. 33);
   the family of -CD79ACD79B-EDRs, which are identical to those described in 1a, 2a, 3a, 4a, 5a, and 6a except that the first instance of the signalling portion of human CD247 is substituted by that of human CD79A (SEQ ID NO. 34) and the second instance of the signalling portion of human CD247 is substituted by that of human CD79B (SEQ ID NO. 35).

EDRs are herein named according to the formula:

A-b₁c₁123/b₂c₂123-EDR
wherein:
   A denotes the TCR from which the EDR's α and β variable domains derive;
   b, where present, denotes the co-stimulation signalling motif b may be absent. b₁ and b₂ may be the same or different;
   c denotes the signalling tail. c₁ and c₂ may be the same or different;
   1, 2 and 3 denote ITAMs within the intracellular portion. Where all three ITAMs are present, ‘1’, ‘2’ and ‘3’ may be omitted from the name. Where one or more ITAMs are ablated, for example by mutation such as Y>F mutation, the corresponding number of ITAM/s is replaced with ‘X’. For example, ‘12X’ denotes ablation of the third ITAM, and ‘1XX’ denotes ablation of the second and third ITAMs.

TABLE 2
Feature key for EDR naming

| | | | SEQ ID NO or |
|---|---|---|---|
| Feature | Name | Description | Identifier |
| A | 1G4 | α and β variable domains | 1 and 4 |
| | | derived from the 1G4 TCR | |
| | A6 | α and β variable domains | 17 and 18 |
| | | derived from the A6 TCR | |
| | a3a | α and β variable domains | 19 and 20 |
| | | derived from the a3a TCR | |
| | wt/c51 | α variable domain derived | 1 and 21 |
| | | from 1G4 TCR and β | |
| | | variable domain derived | |
| | | from wt/c51 TCR | |
| | c259/wt | α variable domain derived | 22 and 4 |
| | | from the c259/wt TCR and β | |
| | | variable domain derived | |
| | | from c259/wt TCR | |
| b | 28 | a signalling motif derived | 10 |
| | | from CD28 | |
| | 2 | a co-stimulatory sequence | 26 |
| | | derived from human CD2 | |
| | CD226 | a co-stimulatory sequence | 28 |
| | | derived from human CD226 | |
| | | (DNAM-1) | |
| | CD28H | a co-stimulatory sequence | 29 |
| | | derived from human CD28H | |
| | CD137 | a co-stimulatory sequence | 30 |
| | | derived from human CD137 | |
| | CD278 | a co-stimulatory sequence | 31 |
| | | derived from human CD278 | |
| c | z | a TCRζ chain (CD247) | 8 |
| | | signalling tail | |
| | e | a CD3ε signalling tail | 11 |
| | d | a CD3δ signalling tail | 12 |
| | g | a CD3γ signalling tail | 13 |
| | DAP12 | a human DAP12 signalling | 27 |
| | | region | |
| | FcεRIγ | a human FcεRIγ | 32 |
| | | signalling region | |
| | FcγRIIa | a human FcγRIIa | 33 |
| | | signalling region | |
| | CD79A | a human CD79A signalling | 34 |
| | | region | |
| | CD79B | a human CD79B signalling | 35 |
| | | region | |
| 1, 2 or 3 | 1, 2 or 3 | ITAM | 40 |
| | X | ITAM ablated through Y > F | 42 |

### Example 4—Comparing Peptide Cross-Reactivity of the 1G4 TCR and the 1G4 EDR

The ability of T cells to display high levels of cross-reactivity is intimately linked to their ability to discriminate antigens based on the TCR/pMHC off-rate (or affinity). As with all protein/protein interactions, the structural mechanisms determining the TCR/pMHC affinity depend on the size of the binding interface and its surface-shape and electrostatic complementarity. Low affinity TCR/pMHC interactions are more probable than high affinity interactions, as they can be achieved by a larger number of structural mechanisms. It follows that the majority of cross-reactive pMHC ligands are likely to bind to the TCR with lower affinity. Indeed, in documented examples of cross-reactivity of wild-type or engineered TCRs, cross-reactivity is commonly observed to lower-affinity pMHCs. It follows that enhanced antigen discrimination will result in lower T cell cross-reactivity.

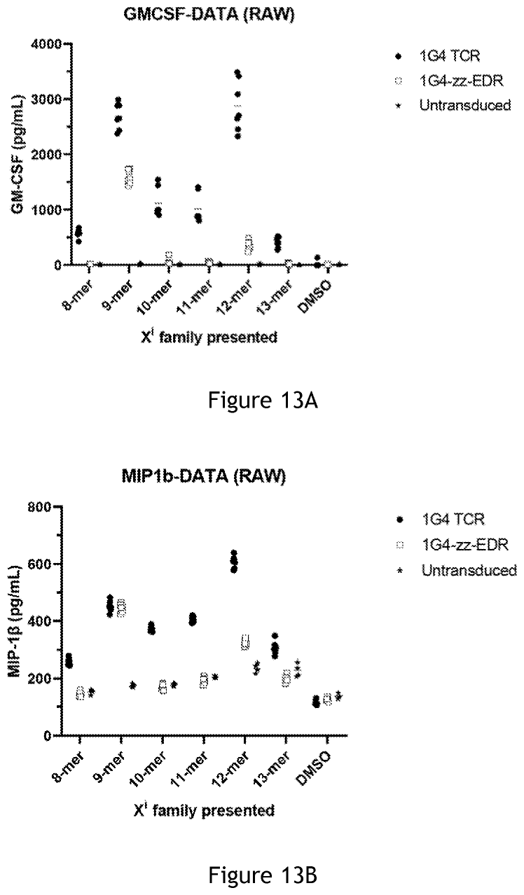

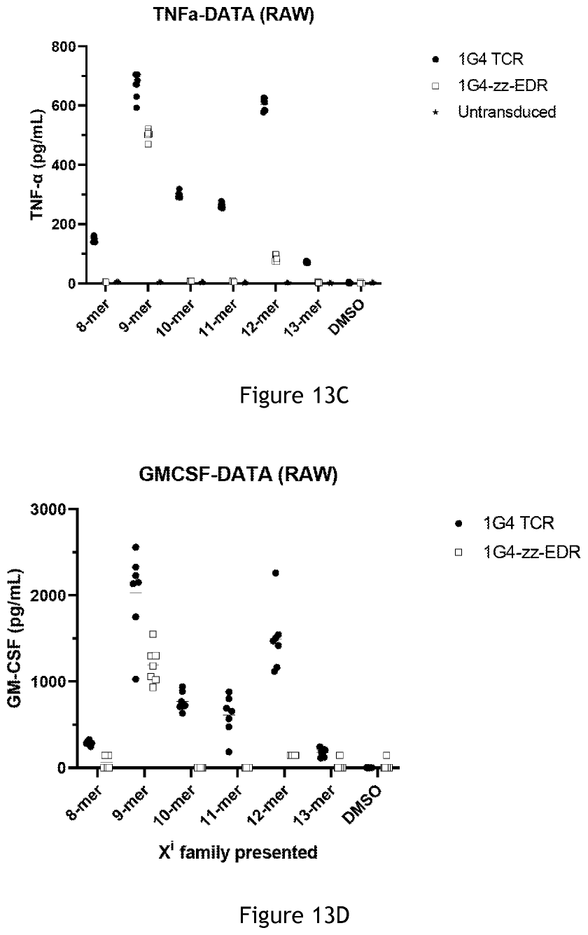

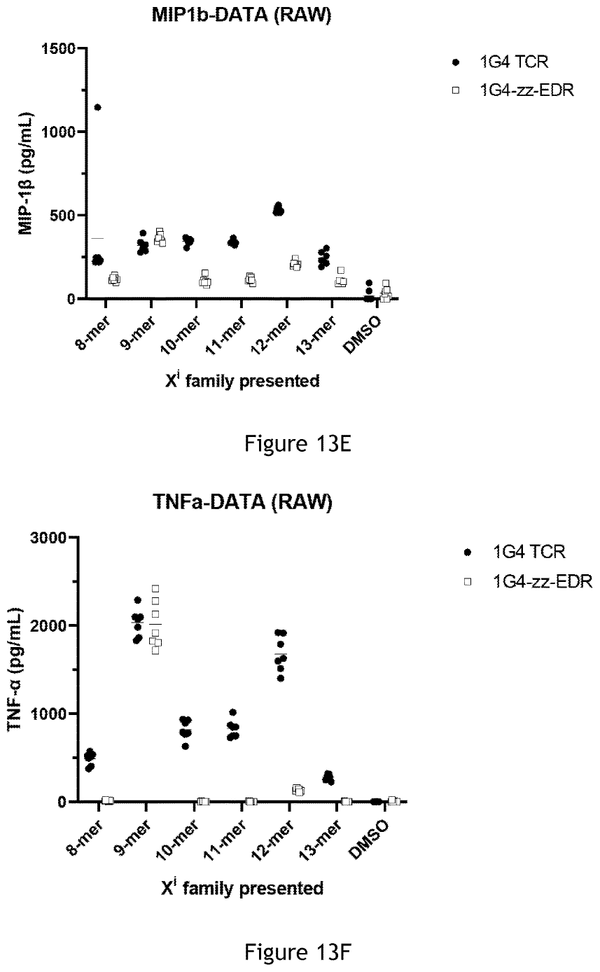

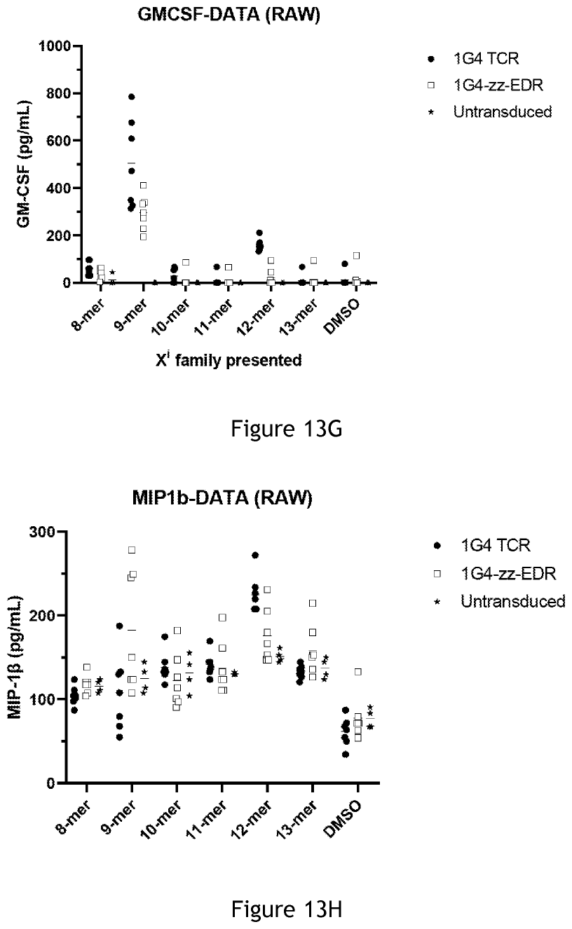

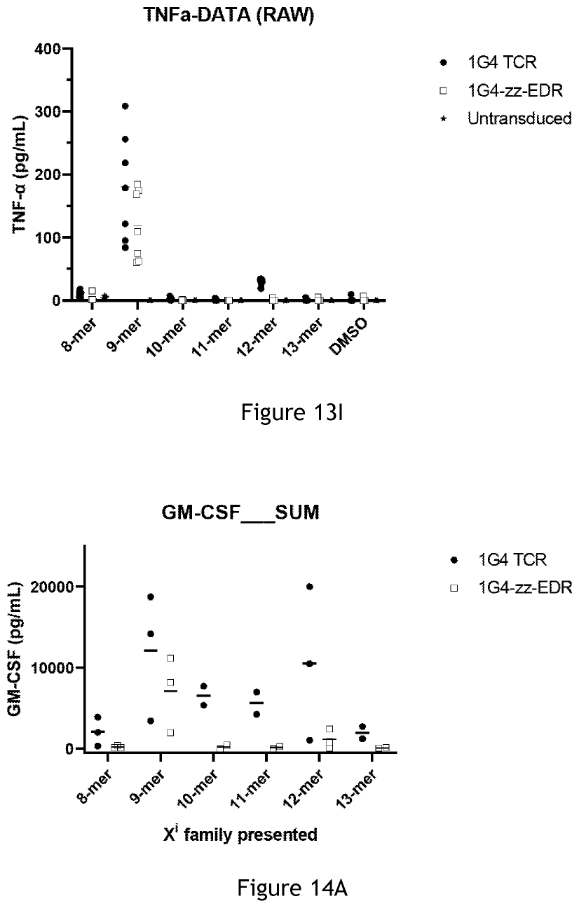

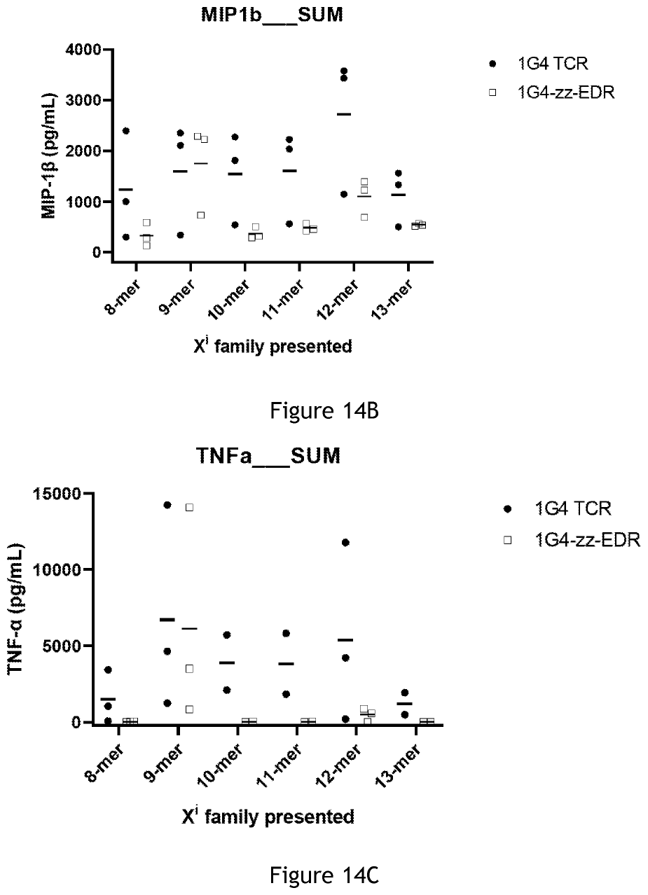

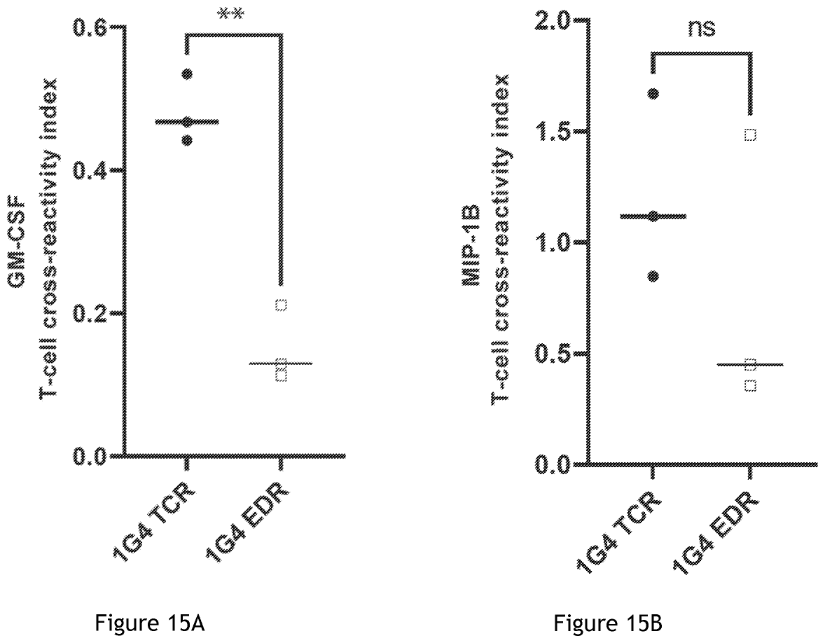

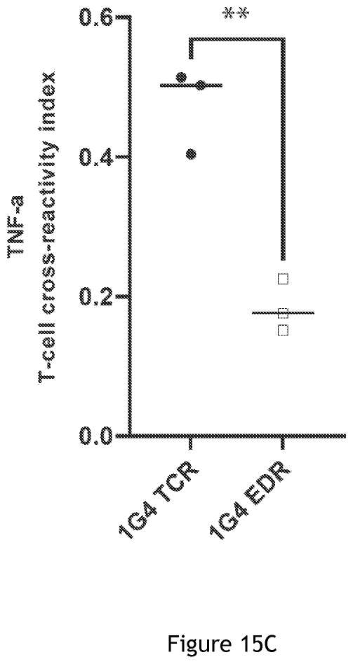

To directly test this, the 1G4 TCR and the 1G4 EDR were co-cultured with libraries containing a random mixture of peptides of defined lengths (X₁X₂ . . . X_{N}, where X is any amino acid except cysteine and N is the length). T cells expressing each receptor were stimulated with T2 cells incubated with these random libraries of N=8, 9, 10, 11, 12, and 13 for 24 hours before measuring the supernatant concentration of three cytokines across 7 technical replicates (FIG. 13). Within each donor, the 1G4 EDR generally produced less cytokine compared to the 1G4 TCR across the different length libraries. Next the cytokine produced across the technical replicates for each donor was summed to obtain an overall measure of cytokine released (FIG. 14) and averaged across the different libraries in order to obtain an index of T cell cross-reactivity (FIG. 15). This index showed that the 1G4 EDR was significantly less cross-reactive compared to the 1G4 TCR for 2 of 3 cytokines tested.

### Material and Methods

### Mixtures of Proteogenic L-Amino Acids

Six different mixtures of proteogenic L-amino acids were ordered from a commercial supplier (Peptide Protein Research Ltd). The identities of each mixture were: XXXXXXXX (i.e. ‘X₈’), X₉, X₁₀, X₁₁, X₁₂ and X₁₃, with X representing any of the 19 proteogenic amino acids excluding cysteine. Thus, X may be R, D, E, S, N, Q, G, P, A, Y, H, I, L, K, M, F, T, W or V. The lyophilised mixtures were resuspended in DMSO to a weighted average concentration of 10 mM and used for cellular experiments.

Experimental Assessment of the IG4 TCR and IG4-z z-EDR Global Cross-Reactivities

The TAP-deficient, HLA-A2:01+T2 cell line was used as APCs to assess the cross-reactivity of the 1G4 TCR and the 1G4-z/z-EDR to the six different mixtures (X⁸ to X¹³) of amino acids. The T2 cells were resuspended at 1×10⁶ cells/mL in full RPMI-1640 and, for each individual mixture, peptide was added to this solution so as to achieve a final concentration of 200 μM of each mixture. In parallel, primary human CD8+ T cells expressing either: (i) no construct (untransduced cells), (ii) the 1G4 TCR, or (iii) the 1G4-z/z-EDR were also resuspended at a concentration of at 1×10⁶ cells/mL in full RPMI-1640.

Afterwards, and for individual each mixture, 100 μL of T2 cells were mixed with 100 μL of primary T cells. As a result, the effective final concentration of each Xⁱ peptide mixture was 100 μM. Cells were plated in 96 well U-bottom plates, lightly centrifuged (20×g during 3 min), and incubated during 24 h in a 37° C. 5% CO₂ (v/v) atmosphere. Following this co-culture, supernatants were assayed for GM-CSF, TNF-α, and MIP-1β. These assays were conducted using commercial human cytokine uncoated ELISA kits from Thermo Fisher Scientific, and the manufacturer's instructions were followed.

Any given combination between T2 cells and cells containing the 1G4 TCR or the 1G4-z/z-EDR was reproduced through three individual donors (each one with seven technical replicates). The combination between T2 cells and untransduced cells was reproduced through two individual donors (each one with four technical replicates).

### Criteria for Data Processing

To compare the data across independent human donors displayed in FIG. 13, the averaged response to DMSO control was subtracted from the experimental results; resultant negative values were assigned a value of zero. Any X-mer family where both the TCR and EDR did not produce cytokine (determined by a one-sample t-test for the null hypothesis that the response was different from 0) were excluded because the difference between TCR and EDR could not be calculated with confidence in these cases. This exclusion criteria resulted in removal of response against 10-, 11-, and 13-mers of donor 3.

### Calculation of the T-Cell Cross-Reactivity Index

To determine a global cross-reactivity index that would capture the response of the 1G4 TCR and the 1G4-z/z-EDR to the whole family of X-mers (X₈ to X₁₃), the aggregated cytokine production (FIG. 14) was normalized, on a per-donor and per-cytokine basis, to the response produced by the 1G4 TCR when presented with the X₉ peptide mixture. The normalised data was then averaged across all X-mer families to obtain the T cell cross-reactivity index for the TCR and EDR for each cytokine per donor.

### REFERENCES

1 Johannes Pettmann, et al “The discriminatory power of the T cell receptor” eLife 10:e67092 (2021) https://doi.org/10.7554/eLife.67092
2 Linette, Gerald P et al. “Cardiovascular toxicity and titin cross-reactivity of affinity-enhanced T cells in myeloma and melanoma.” Blood vol. 122, 6 (2013): 863-71.
3 Cameron, Brian J et al. “Identification of a Titin-derived HLA-A1-presented peptide as a cross-reactive target for engineered MAGE A3-directed T cells.” Science translational medicine vol. 5,197 (2013): 197ra103.
4 Michael S. Kuhns, et al. “Deconstructing the Form and Function of the TCR/CD3 Complex” Immunity, 24(2), Pages 133-139, (2006)
5 Rurik et al., Science 375(6576):91-96 (2022).

Brief description of the sequences

| SEQ | |
|---|---|
| ID NO: | Description |
| 1 | 1G4 TCR Variable alpha region |
| 2 | 1G4 TCR constant alpha region |
| 3 | P2A self-cleaving peptide |
| 4 | 1G4 TCR Variable beta region, |
| 5 | 1G4 TCR constant beta region |
| 6 | TCR Truncated constant alpha region |
| 7 | CD28 transmembrane region |
| 8 | TCRζ (CD247) signalling tail |
| N/A | Glycine-serine linker |
| 9 | TCR Truncated constant beta region |
| 10 | CD28 signalling motif |
| 11 | CD3ε signalling tail |
| 12 | CD3δ signalling tail |
| 13 | CD3γ signalling tail |
| 14 | Modified CD247 chain wherein third ITAM |
| | is ablated through Y > F |
| 15 | Modified CD247 chain wherein second and |
| | third ITAMs are ablated through Y > F |
| 16 | Modified CD247 chain wherein first, second |
| | and third ITAMs are ablated through Y > F |
| 17 | A6 TCR variable alpha domain |
| 18 | A6 TCR variable beta domain |
| 19 | a3a TCR variable alpha domain |
| 20 | a3a TCR variable beta domain |
| 21 | Wt/c51 TCR variable beta domain |
| 22 | C259/wt TCR variable alpha domain |
| 23 | Human CD8alpha transmembrane sequence |
| 24 | Human CD4 TM sequence |
| 25 | Human CD247 TM sequence |
| 26 | Human CD2 co-stimulatory sequence |
| 27 | Signalling portion of human DAP12 |
| 28 | Human CD226 (DNAM-1) co-stimulatory sequence |
| 29 | CD28H co-stimulatory sequence |
| 30 | CD137 co-stimulatory sequence |
| 31 | CD278 co-stimulatory sequence |
| 32 | Signalling portion of human FcεRIγ |
| 33 | Signalling portion of human FcγRIIa |
| 34 | Signalling portion of human CD79A |
| 35 | Signalling portion of human CD79B |
| 36 | IgG1-CHI domain |
| 37 | IgG1-CL domain |
| 38 | Sequence removed from 1G4 TCR α constant |
| | region to minimise CD3 complexation |
| 39 | Sequence removed from 1G4 TCR β constant |
| | region to minimise CD3 complexation |
| 40 | conserved ITAM motif |
| 41 | conserved hemITAM motif: YxxL, wherein x |
| | is any amino acid |
| 42 | ITAM ablated through Y > F |
| 43 | β-CPM of T cell receptor beta constant |
| | variant 1 (TRBC1, UniProt P01850) |
| 44 | β-CPM of T cell receptor beta constant |
| | variant 2 (TRBC2, UniProt (UniProt A0A5B9) |
| 45 | α-CPM |
| 46 | β-CPM |
| 47 | Consensus sequence of highly conserved |
| | charged residues within the TCR alpha |
| | chain transmembrane region |
| 48 | Shared consensus sequence of highly |
| | conserved charged residues within the |
| | TCR beta chain transmembrane region |
| 49 | 1G4 TCR alpha chain transmembrane region |
| 50 | 1G4 TCR alpha chain cytoplasmic tail |
| 51 | 1G4 TCR beta chain transmembrane region |
| 52 | 1G4 TCR beta chain cytoplasmic tail |
| 53 | 1G4 T cell receptor coding sequence |
| 54 | 1G4-z/z-EDR |
| 55 | 1G4-28z/28z-EDR |
| 56 | 1G4-z/z-EDR, Alpha Chain |
| 57 | 1G4-z/z-EDR, Beta Chain |
| 58 | 1G4-28z/28z-EDR, Alpha Chain |
| 59 | 1G4-28z/28z-EDR, Beta Chain |
| 60 | c259/wt-z/z-EDR, Alpha Chain |
| 61 | c259/wt-28z/28z-EDR, Alpha Chain |
| 62 | a3a-z/z-EDR, Alpha Chain |
| 63 | a3a-z/z-EDR, Beta Chain |
| 64 | a3a-28z/28z-EDR, Alpha Chain |
| 65 | a3a-28z/28z-EDR, Beta Chain |
| 66 | CPM conserved motif: CXXC, wherein |
| | X is any amino acid |
| 67 | 1G4 TCR constant alpha region, |
| | transmembrane, and cytoplasmic tail |
| 68 | 1G4 TCR Constant beta region, |
| | transmembrane, and cytoplasmic tail |
| 69 | Transmembrane region of T cell receptor beta |
| | constant variant 1 (TRBC1, UniProt P01850) |
| 70 | Transmembrane region of T cell receptor beta |
| | constant variant 2 (TRBC2, UniProt A0A5B9) |

1. A MHC-presented peptide (pMHC)-binding receptor comprising two polypeptides forming a heterodimer which comprises: (a) an extracellular portion comprising a pMHC-binding portion derived from a T cell receptor (TCR); (b) a transmembrane portion; and (c) an intracellular portion comprising a signalling domain.

2. The pMHC-binding receptor of claim 1, wherein:
(i) the first polypeptide comprises: (a) an extracellular portion derived from a TCR; (b) a transmembrane portion; (c) an intracellular portion; and
(ii) the second polypeptide comprises: (a) an extracellular portion derived from a TCR; (b) a transmembrane portion; (c) an intracellular portion.

3. The pMHC-binding receptor of claim 1 or 2, wherein:
the extracellular portion of the first polypeptide comprises: (a) a TCR CDR1α loop, a TCR CDR2α loop and a TCR CDR3α loop; and or (b) a TCR α variable domain,
the extracellular portion of the second polypeptide comprises: (a) a CDR1β loop, a CDR2β loop and a CDR3β loop; and/or (b) a TCR β variable domain; and
wherein complexation of the two variable domains forms the pMHC-binding portion in the extracellular portion of the pMHC-binding receptor.

4. The pMHC-binding receptor of any one of claims 1 to 3, wherein the extracellular portion of the pMHC-binding receptor further comprises a structural portion which is membrane proximal to the pMHC-binding portion and is formed upon complexation of the two polypeptides.

5. The pMHC-binding receptor of claim 4, wherein the structural portion is derived from a T cell receptor.

6. The pMHC-binding receptor of claim 5, wherein the extracellular portion of the first polypeptide comprises a truncated TCR α constant region (e.g SEQ ID NO: 6), and the extracellular portion of the second polypeptide comprises a truncated TCR β constant region (e.g. SEQ ID NO: 9), and complexation of the constant regions form the membrane-proximal structural portion in the extracellular portion of the pMHC-binding receptor.

7. The pMHC-binding receptor of claim 4, wherein the structural portion is derived from an antibody.

8. The pMHC-binding receptor of claim 7, wherein the extracellular portion of the first polypeptide comprises the heavy chain constant domain of an antibody (e.g. IgG1-CH1 domain, such as SEQ ID NO: 36), and the extracellular portion of the second polypeptide comprises the light chain constant domain of the antibody (e.g. SEQ ID NO: 37), and complexation of the constant domains form the membrane proximal structural portion in the extracellular portion of the pMHC-binding receptor.

9. The pMHC-binding receptor of any one of claims 1 to 8, wherein the intracellular portion of the first and/or second polypeptide comprises a signalling domain comprising one or more pairs of immunoreceptor tyrosine-based activation motifs (ITAMs) (e.g. SEQ ID NO: 40) or one or more hemITAMs (e.g. SEQ ID NO: 41).

10. The pMHC-binding receptor of any one of claims 1 to 9, wherein the signalling portion of the first and/or second polypeptide comprises two or more pairs of ITAMs, optionally three pairs of ITAMs.

11. The pMHC-binding receptor of any one of claims 1 to 10, wherein the intracellular portion of the first and/or second polypeptide comprises the intracellular portion of one or more TCRζ chains (e.g. SEQ ID NO: 8).

12. The pMHC-binding receptor of any one of claims 1 to 11, wherein the transmembrane portion of the first and/or second polypeptide comprises the transmembrane portion of CD28 (e.g. SEQ ID NO: 7) or CD8α (e.g. SEQ ID NO: 23).

13. The pMHC-binding receptor of any one of claims 1 to 12, wherein the intracellular portion of the first and/or second polypeptide additionally comprises one or more costimulatory domains, optionally wherein the one or more costimulatory domains comprises any of CD2 (SEQ ID NO: 26), CD226 (SEQ ID NO: 28), CD28H (SEQ ID NO: 29), and/or CD137 (SEQ ID NO: 30) costimulatory domains.

14. The pMHC-binding receptor of any one of claims 1 to 13, wherein the pMHC-binding receptor does not associate with a CD3 complex.

15. The pMHC-binding receptor of claim 14, wherein the extracellular portion of the first polypeptide does not comprise an amino acid sequence having sequence SEQ ID NO: 45 and the second polypeptide does not comprise an amino acid sequence having sequence 46; and/or wherein the transmembrane portion of the first and/or second polypeptide does not comprise an amino acid sequence SEQ ID NO: 47 and/or 48 respectively.

16. The pMHC-binding receptor of any one of claims 1 to 15, wherein:
(i) the first polypeptide comprises: (a) an extracellular portion comprising a TCR α variable domain and a truncated TCR α constant region (e.g. SEQ ID NO: 6); (b) a transmembrane region from CD28 (e.g. SEQ ID NO: 7) or CD8α (SEQ ID NO: 23); and (c) an intracellular region comprising a signalling domain from TCR ζ (e.g. SEQ ID NO: 8); and
(ii) the second polypeptide comprises: (a) an extracellular region comprising a TCR β variable domain and a truncated TCR β constant region (e.g. SEQ ID NO: 9); (b) a transmembrane region from CD28 (e.g. SEQ ID NO: 7) or CD8α (SEQ ID NO: 23); and (c) an intracellular region comprising a signalling domain from TCRζ (e.g. SEQ ID NO: 8),
optionally wherein the intracellular region of each of the first and the second polypeptides further comprises any of CD2 (SEQ ID NO: 26), CD226 (SEQ ID NO: 28), CD28H (SEQ ID NO: 29), and/or CD137 (SEQ ID NO: 30) costimulatory domains.

17. The pMHC-binding receptor of claim 15 or 16, wherein each TCRζ chain comprises at least one ITAM.

18. The pMHC-binding receptor of claim 17, wherein each TCRζ chain comprises at least two ITAMs, optionally three ITAMs.

19. The pMHC-binding receptor of any one of claims 1 to 18 wherein the pMHC-binding receptor exhibits enhanced discrimination for its target antigen relative to a TCR which binds the same target antigen.

20. The pMHC-binding receptor of any one of claims 1 to 19 wherein, when expressed in a cell, the pMHC-binding receptor is surface-presented.

21. One or more isolated polynucleotides encoding the first polypeptide of the pMHC-binding receptor as defined in any one of claims 1 to 20.

22. One or more isolated polynucleotides encoding the second polypeptide of the pMHC-binding receptor as defined in any one of claims 1 to 20.

23. One or more isolated polynucleotides encoding the first polypeptide and the second polypeptide of the pMHC-binding receptor as defined in any one of claims 1 to 20.

24. One or more expression constructs comprising one or more polynucleotides as defined in any one of claims 21 to 23.

25. One or more vectors comprising the one or more polypeptides of any one of claims 21 to 23 or the one or more expression constructs of claim 24.

26. A cell comprising one or more polynucleotides as defined in any one of claims 21 to 23, one or more expression constructs as defined in claim 24, or one or more vectors as defined in claim 25.

27. A cell comprising a pMHC-binding receptor as defined in any one of claims 1 to 21.

28. The cell of claim 27, wherein the cell is an immune cell, optionally an immune effector cell.

29. The cell of claim 28, wherein the immune cell is a T lymphocyte, optionally an inflammatory T lymphocyte, a cytotoxic T lymphocyte, a regulatory T lymphocyte, or a helper T lymphocyte.

30. The cell of claim 29, wherein the T lymphocyte is a CD8+ cytotoxic T lymphocyte.

31. A pharmaceutical composition comprising: (i) one or more polynucleotides as defined in any one of claims 21 to 23, one or more expression constructs as defined in claim 24, or one or more vectors as defined in claim 25, and (ii) a pharmaceutically acceptable carrier.

32. A pharmaceutical composition comprising a cell as defined in any one of claims 26 to 30 and a pharmaceutically acceptable carrier.

33. A pMHC-binding receptor as defined in any one of claims 1 to 20, one or more polynucleotides as defined in any one of claims 21-23, one or more expression constructs as defined in claim 24, or one or more vectors as defined in claim 25, or a cell as defined in any one of claims 26 to 30 for use as a medicament.

34. A pMHC-binding receptor as defined in any one of claims 1 to 20, one or more polynucleotides as defined in any one claims 21 to 23, one or more expression constructs as defined in claim 24, or one or more vectors as defined in claim 25, or a cell as defined in any one of claims 26 to 30 for use in treating cancer.

35. A pMHC-binding receptor as defined in any one of claims 1 to 20, one or more polynucleotides as defined in any one claims 21 to 23, one or more expression constructs as defined in claim 24, or one or more vectors as defined in claim 25, or a cell as defined in any one of claims 26 to 30 for use in treating an infection, optionally a chronic infection, or an inflammatory disease, optionally an autoimmune disease.

36. Use of a pMHC-binding receptor as defined in any one of claims 1 to 20, one or more polynucleotides as defined in any one claims 21 to 23, one or more expression constructs as defined in claim 24, or one or more vectors as defined in claim 25, or a cell as defined in any one of claims 26 to 30 in the manufacture of a medicament for the treatment of cancer.

37. Use of a pMHC-binding receptor as defined in any one of claims 1 to 20, one or more polynucleotides as defined in any one of claims 21 to 23, one or more expression constructs as defined in claim 24, or one or more vectors as defined in claim 25, or a cell as defined in any one of claims 26 to 30 in the manufacture of a medicament for the treatment of an infection, optionally a chronic infection, or an inflammatory disease, optionally an autoimmune disease.

38. A method of treating cancer comprising administering a pMHC-binding receptor as defined in any one of claims 1 to 20, one or more polynucleotides as defined in any one of claims 21 to 23, one or more expression constructs as defined in claim 24, or one or more vectors as defined in claim 25, or a cell as defined in any one of claims 26 to 30 to a patient in need thereof.

39. A method of treating an infection, optionally a chronic infection, or an inflammatory disease, optionally an autoimmune disease, comprising administering a pMHC-binding receptor as defined in any one of claims 1 to 20, one or more polynucleotides as defined in any one of claims 21 to 23, one or more expression constructs as defined in claim 24, or one or more vectors as defined in claim 25, or a cell as defined in any one of claims 26 to 30 to a patient in need thereof.

40. Use of the pMHC-binding receptor as defined in any one of claims 1 to 20, one or more polynucleotides as defined in any one of claims 21 to 23, one or more expression constructs as defined in claim 24, or one or more vectors as defined in claim 25, or a cell as defined in any one of claims 26 to 30 to treat cancer.

41. Use of the pMHC-binding receptor as defined in any one of claims 1 to 20, one or more polynucleotides as defined in any one of claims 21 to 23, one or more expression constructs as defined in claim 24, or one or more vectors as defined in claim 25, or a cell as defined in any one of claims 26 to 30 to treat an infection, optionally a chronic infection, or an inflammatory disease, optionally an autoimmune disease.

42. A method of identifying a pMHC-binding receptor specific to a target pMHC of interest, wherein the method comprises screening a library of pMHC-binding receptors of any one of claims 1 to 20 for high affinity binding to said target pMHC.

43. A method of identifying low-affinity or ‘off-target’ binding interactions comprising screening a library of MHC-presented peptides (pMHCs) for binding to a pMHC-binding receptor of any one of claims 1 to 20, optionally wherein said library does not comprise the pMHC-binding receptor's target pMHC.

44. A method of assessing discrimination capacity of a pMHC-binding receptor of any one of claims 1 to 20, comprising exposing the pMHC-binding receptor to on-target higher affinity and one or more off-target low affinity pMHCs (i.e. pMHC for which it is not specific) and comparing the concentration of pMHC required to induce activation of cells expression the pMHC-binding receptor, wherein a large difference in concentration indicates good discrimination.

45. The method of any one of claims 42 to 44 wherein the method is performed ex vivo.

46. A molecule obtainable by the method of any one of claims 42 to 44.

47. A method, such as an ex vivo method, of preparing a population of immune cells for adoptive cell therapy, the method comprises culturing the immune cell of any one of claims 28 to 30 to produce a population of immune cells.

48. A population of immune cells produced by the method of claim 47.
